SEQUENCE LISTING (2 sequences)

SEQ ID NO: 1 - protein, 24 aa, Artificial sequence
  secrection signal peptide
MKLKRLMAAMTFVAAGVATANAVA

SEQ ID NO: 2 - protein, 32 aa, Artificial sequence
  secretion signal peptide
MPTTPHSFHLSPQGKLRWAIASLFLLPQLALA

(19) United States
(12) Patent Application Publication
(10) Pub. No.: US 2007/0238153 A1
(43) Pub. Date: Oct. 11, 2007

(54) Processes for improved disulfide bond formation in recombinant systems

(75) Inventor: Charles H. Squires, Poway, CA (US)

Correspondence Address: TRASK BRITT, P.O. BOX 2550, SALT LAKE CITY, UT, 84110, US

(73) Assignee: Dow Global Technologies Incorporated, Midland, MI (US)

(21) Appl. No.: 11/784,237

(22) Filed: Apr. 6, 2007

Related U.S. Application Data
(60) Provisional application No. 60/790,059, filed on Apr. 7, 2006.

(51) Int. Cl. C12P 21/06; C07H 21/04; C12N 9/02; C12N 1/21; C12N 15/74

(52) U.S. Cl. 435/69.1; 435/189; 435/252.34; 435/471; 536/23.2

(57) ABSTRACT

Methods and prokaryotic expression systems for producing recombinant disulfide bond-containing proteins are described including increasing the level of at least one redox cofactor in the cell to enhance disulfide bond formation are disclosed. Processes of increasing levels of redox cofactors include media supplementation and genetic modification of the host cell. The process can yield increased levels of soluble or active protein, or can assist the proper processing of recombinant proteins in expression systems.

### CROSS-REFERENCE TO RELATED APPLICATION

This application claims the benefit of U.S. Provisional application No. 60/790,059, filed Apr. 7, 2006, entitled “Processes For Improved Disulfide Bond Formation In Recombinant Systems.”

### FIELD OF THE INVENTION

This present invention is in the field of protein production and relates to an improved method to produce properly folded and active heterologous proteins containing disulfide bonds by increasing the levels of redox cofactors in the cells during expression of recombinant peptides.

### BACKGROUND

More than 150 recombinantly produced proteins and peptides have been approved by the U.S. Food and Drug Administration (FDA) for use as biotechnology drugs and vaccines, with another 370 in clinical trials. Unlike small molecule therapeutics that are produced through chemical synthesis, proteins and peptides are most efficiently produced in living cells. However, current methods of production of recombinant proteins in bacteria often produce improperly folded, aggregated, or inactive proteins, and many types of proteins require secondary modifications that are inefficiently achieved using known methods.

Post-Translational Modifications

Many proteins require certain post-translational modifications for proper folding or activity. A key step in protein folding is often the formation of disulfide bonds between cysteine residues. Disulfide bonds frequently play an essential structural role within proteins, stabilizing their tertiary structures. The reduction of disulfide bonds can lead to protein unfolding. Unfortunately, the most commonly used expression systems do not promote proper formation of disulfide bonds in recombinantly expressed proteins.

Post-translational modifications represent one of the differences between bacterial and eukaryotic protein expression. Expression of multiple disulfide bond-containing eukaryotic polypeptides, and particularly mammalian proteins, in bacterial cells has produced disappointing and unsatisfactory results. Bacteria can form disulfide bonds. However, refolding is usually required because the bonds are not always formed in the correct configuration required for biological activity. Correct folding may depend on the formation of cysteine-cysteine linkages and subsequent stabilization of the protein into an enzymatically active structure.

Disulfide Bond Assembly

Certain molecular chaperones can catalyze protein folding by assisting the self-assembly process. They can function by binding to and stabilizing unfolded or partially folded polypeptides that are intermediates along the pathway leading to the final correctly folded state. The first of these protein disulfide isomerase (PDI) was discovered in 1963 in the eukaryotic endoplasmic reticulum (Goldberger, et al. (1963) J. Biol. Chem. 238, 628-635). In eukaryotes and prokaryotes, disulfide bond oxidation, reduction, and isomerization are catalyzed processes, facilitated by members of the thioredoxin superfamily, as well as those of the disulfide bond oxidoreductase isomerase family, such as the dsbA, B, C, D and G genes of E. coli.

Although disulfide bonds are essential stabilizing structures in many proteins, they are rarely found in the cytoplasm of prokaryotic or eukaryotic organisms. This is because the cyoplasmic environment is too reduced to allow these bonds to remain oxidized. Instead, disulfide bonds are usually found in proteins destined for locations outside of the cytoplasm. In prokaryotes, the disulfide bonds can be formed within the periplasm. This may be due to several cellular needs, including that a number of cytoplasmic enzymes rely on a reduced cysteine residue in their active site and that a partially unfolded conformation is required for the translocation of many proteins across membranes. To ensure that disulfide bond formation only occurs in the specialized locations, prokaryotes have evolved impediments to disulfide bond formation in the cytoplasm, such as a reducing environment in the cytoplasm.

Proteins that are capable of catalyzing protein disulfide bond formation are members of a large collection of thiol-disulfide oxidoreductases found in all living cells. Many of these enzymes belong to the thioredoxin superfamily, which is defined by an active site containing a CXXC motif (cysteines separated by two amino acids) and by a thioredoxin fold seen in the three-dimensional structure of the prototypical thioredoxin 1 of E. coli. While in extracytoplasmic compartments these proteins can act as oxidants, those located in the cytoplasm perform mainly reductive steps. One of the cytoplasmic activities for many bacteria is the reduction of ribonucleotide reductase (an essential enzyme that converts ribonucleotides to deoxyribonucleotides) by thioredoxins and glutaredoxins.

Other enzymes have been identified that are not members of the thioredoxin superfamily but which also use redox active cysteines in transferring electrons in oxidative and reductive pathways. Typically, these enzymes use redox active cysteine pairs that are separated by more than two amino acids. In addition, these enzymes may use small molecule electron donor and receptor cofactors, such as FAD, NADPH, NADH, quinones, and lipoic acid (Bryk, et al. (2002) Science 295:1073-77). Many of these nonthioredoxin-like enzymes themselves receive from or donate electrons to proteins belonging to the thioredoxin-like class. For example, the protein DsbB does not contain a thioredoxin fold, but transfers electrons via two pairs of redox active cysteines from the thioredoxin-like DsbA to quinones.

Disulfide Bond Formation

In the E. coli periplasm, disulfide bond formation and disulfide bond isomerization are catalyzed by two pathways, as shown in FIG. 1. The DsbA-DsbB pathway oxidizes thiol groups to form disulfides de novo, while the DsbC-DsbD pathway isomerizes mismatched disulfides. In the oxidative pathway, DsbA directly interacts with substrate proteins and oxidizes them. DsbA is a 21 kDa periplasmic protein that contains a thiredoxin fold, as well as two active site cysteines residues in a CXXC motif. Unlike disulfide isomerases, DsbA must have an oxidized active site in order to transfer disulfides to newly synthesized proteins.

DsbB's role in the DsbA-DsbB pathway is to reoxidize DsbA, allowing DsbA to regain activity. DsbB becomes reduced after it has reoxidized DsbA. To function catalytically, DsbB must therefore be reoxidized. The electron transport chain appears to be involved in reoxidizing DsbB, as mutants defective in ubiquinone (ubiA-menA mutations) or heme biosynthesis (hemA mutation) accumulate DsbA in reduced form (Bader, et al. (1998) J. Biol. Chem. 273, 10302-7). DsbB has been shown to include the presence of cytochrome oxidases for its activity. These cytochrome oxidases can act as the terminal electron acceptor in the electron transport chain, transferring electrons from ubiquinone to molecular oxygen. DsbC is a periplasmic protein with thiol-disulfide oxidoreductase activity both in vivo and in vitro (Missiakas, et al. (1994) Embo J. 13, 2013-20; Shevchik, et al. (1994) Embo J. 13, 2007-12). DsbC is a member of the thioredoxin family whose members include DsbA, PDI, and thioredoxin. DsbC can rearrange incorrectly formed disulfides both in vitro and in vivo. In the presence of an oxidant (either oxidized glutathione or DsbA), DsbC increases the rate of formation of native protein with disulfide bond formation, without increasing the rate of disappearance of the fully reduced protein. This occurs because DsbC causes disulfide rearrangements in the stable misfolded intermediates of proteins, such as bovine pancreatic trypsin inhibitor (BPTI), allowing the native disulfide pairings to occur. (Zapun, et al. (1995) Biochemistry 34, 5075-89.) DsbG is a second putative disulfide isomerase. The inner membrane protein DsbD reduces DsbC and DsbG. As electrons are passed from NADPH to thioredoxin via thioredoxin reductase, periplasmic DsbC is kept reduced by DsbD at the expense of NADPH oxidation in the cytoplasm.

It is possible to observe protein disulfide bond formation in the cytoplasm of E. coli without changing the overall reductive nature of this compartment by mutationally altering certain electron transfer pathways. (Stewart, et al. (1998) EMBO J. 17:5543-50.) These alterations lead to the accumulation of oxidized thioredoxins, which then act as catalysts of disulfide bond formation.

Numerous attempts have been developed to increase production of properly folded proteins in recombinant systems. For example, investigators have changed fermentation conditions (Schein (1989) Bio/Technology, 7:1141-1149), varied promoter strength for induction of recombinant protein expression, or used overexpressed chaperone proteins (Hockney (1994) Trends Biotechnol. 12:456-463), which can help prevent the formation of inclusion bodies.

U.S. Pat. No. 5,077,392 discloses a renaturation method for refolding denatured proteins obtained after expression in inclusion bodies. tPA was isolated as a denatured reduced protein and subsequently refolded under oxidizing conditions, which could allow disulfide bond formation, to obtain what was reported as up to a 26% yield of “reactivated” protein. While the method appeared to improve polypeptide yield, the process involves multiple, time-consuming steps, due to the initial recovery of the insoluble, inactive protein.

Numerous groups have used increased periplasmic secretion and/or increased expression of disulfide isomerases to produce properly folded and/or active protein in hosts. For example, U.S. Pat. No. 6,083,715 to the Board of Regents, The University of Texas and Genentech, describes the production of heterologous polypeptide containing many disulfide bonds in bacteria that also express a disulfide isomerase to ensure proper folding and increased yield of biologically active product. Tissue plasminogen activator and pancreatic trypsin inhibitor are provided as examples.

U.S. Pat. No. 6,027,888 to the Board of Regents, The University of Texas describes bacterial production of biologically active, soluble, disulfide-bonded eukaryotic proteins via co-expression of a eukaryotic disulfide isomerase. The patent describes this co-expression as useful for production of, for example, pancreatic trypsin inhibitor and tissue plasminogen activator.

U.S. Publication No. 2004/0018596 describes a DNA sequence that includes one or two promoters required for gene expression, two Shine-Dalgarno sequences, two identical or different sequences encoding signal peptides of cell wall proteins of a Bacillus bacterium, a gene encoding a polypeptide having disulfide bonds, and a gene encoding protein disulfide isomerase that are ligated to each other. The DNA is described as enabling co-expression of a protein disulfide isomerase and a polypeptide having disulfide bonds and enhancing the efficiency of formation of correct disulfide bonds.

Eur. Pat. Appl. No. EP 510,658 describes an improvement of the yield of secreted disulfide-bonded proteins in bacterial cell by providing a simultaneous expression of a recombinant vector encoding the prokaryotic protein disulfide isomerase of E. coli and the addition of thiol reagents to the culture medium to promote correct folding of the secreted polypeptide of interest.

As none of these methods has been universally effective at improving the efficiency of disulfide bond formation in vivo on a range of disulfide-bonded recombinant proteins, there is still a need in the art for improved large-scale expression systems capable of producing disulfide bonds in recombinant polypeptides to produce transgenic proteins in active form. It is hypothesized that the reason none of these attempts have improved recombinant disulfide bond formation in vivo is that all of the factors, including disulfide bond oxido-reductase isomerase enzymatic activities, as well as their respective redox cofactors, required for these processes to occur have not been coordinately engineered so that none of the required functions becomes rate limiting.

### SUMMARY OF THE INVENTION

The present invention provides processes and compositions for producing properly folded or active recombinant protein containing disulfide bonds by increasing the levels of redox cofactor in a host cell. The level of cofactors can be increased by including the cofactor or a precursor to the cofactor in the cell growth media, or can be increased directly by modifying the host cell genome to result in increased expression of a required cofactor in vivo. In one embodiment, the cell also expresses at least one recombinant disulfide bond isomerase enzyme. When a disulfide bond isomerase is exogenously expressed, typically the level of disulfide bond formation is limited not by availability of the enzyme, but by availability of the cofactors required for oxidation and reduction of the isomerase/oxidoreductase enzymes. By increasing the amount of redox cofactor available for disulfide bond formation, the redox reaction is not rate limiting.

In an embodiment of the invention, a process of improving production of disulfide bonds in a host is provided that includes expressing a recombinant protein that includes at least two cysteine residues in a host cell, and increasing the concentration of a redox cofactor in the host. In one embodiment, the redox cofactor is included in the media that the host is incubated in. In this embodiment, the cofactor can be taken up by the natural mechanisms in the host cell, or the host cell membrane can be made more porous to the cofactor.

In another embodiment, the process includes modifying the host to produce increased levels of a redox cofactor. In some embodiments, this is accomplished through mutation of a gene that is involved in cofactor production or regulation of production. In other embodiments this is accomplished by mutation of a gene that is involved in cofactor degradation.

The level of redox cofactor can be increased throughout the host cell. However, typically, the level is increased in the periplasm of the host cell. In bacterial cells, the periplasm is typically the site of disulfide bond formation and the location for isomerase enzymes.

In some embodiments, the cofactor is a substituted or unsubstituted quinone. In a specific embodiment, the cofactor is ubiquinone or menaquinone. In this embodiment, the cofactor can be included in the media or can be directly produced by the host by also including a recombinant sequence that increases quinone production in the host. The quinone can be a hydroquinone, such as 2,3-dimethoxy-5-methyl-6-decyl-1,4-hydroquinone, which appears to be involved in the stabilization of DsbB. It appears that disulfide bond formation involves a stacked hydroquinone-benzoquinone pair that can be trapped on DsbB as a quinhydrone charge-transfer complex. The quinone can also include a benzoquinone. In other embodiments, the cofactor can be pyrroloquinoline quinone. In some embodiments, both hydrozyquinone and benzoquinone are included to increase the content of quinhydrone.

In another embodiment, the process includes increasing the rate of NADPH production in the cell. NADPH can be increased by direct incubation of the cell with NADPH or can be increased by increasing the metabolic rate of the cell.

In another embodiment, the host cell can be genetically modified to alter expression of a cofactor. In one specific embodiment, the gene encoding a Coq7 enzyme or analog thereof, which can produce ubiquinone, is altered.

In another embodiment, the process includes, along with increased levels of redox cofactors, increasing the expression of disulfide bond isomerase/oxido-reductase enzymes in the host. The isomerase enzymes can be selected from DsbA, DsbB, DsbC, DsbD, or DsbG. In some embodiments, more than one isomerase/oxido-reductase enzyme is increased in expression. The enzyme can be increased by, for example, including a vector that can express the enzyme in the host and inducing expression. The isomerase/oxido-reductase enzyme can be native to the host cell, or can be derived from a different species than the host cell. The isomerase/oxido-reductase enzyme can also be mutated to increase efficiency of binding to a particular protein, or to increase catalytic capacity.

Other embodiments include a process for producing in a bacterial cell, a biologically active, soluble polypeptide of interest having at least one disulfide bond. In a separate embodiment, the native form of the polypeptide of interest contains at least three, four, five, six, seven, eight, nine, ten, eleven, twelve, thirteen, fourteen, fifteen, sixteen, seventeen, eighteen, nineteen, twenty, twenty-five, thirty, thirty-five, fourty or fifty disulfide bonds.

The process can include expressing in the cell a first DNA segment encoding a disulfide isomerase/oxido-reductase operably linked to a signal sequence and a second DNA segment encoding a polypeptide of interest, operably linked to a signal sequence under conditions effective to produce the polypeptide of interest. In some embodiments, the polypeptide can be a eukaryotic polypeptide. In others, it can be derived from a prokaryotic species. The polypeptide can be a mammalian polypeptide. In further embodiments, the polypeptide can be a human polypeptide. The protein or peptide can be a monomer or can be a concatameric peptide. In some embodiments of concatameric proteins, the concatamers can also include linker groups, which can be cleaved using known methods.

In one embodiment, the recombinant polypeptide of interest is expressed and/or transported to the periplasm of the host cell. In other embodiments of the present invention, the polypeptide can contain a secretion signal sequence that targets the periplasm of the cell. In further embodiments this sequence is derived from a Pseudomonas fluorescens organism and can include, for example, a sequence derived from an Outer Membrane Porin E (OprE) secretion signal, a Lys-Arg-Orn binding protein secretion signal, an azurin secretion signal, an iron (III) binding protein secretion signal, a lipoprotein B secretion signal or a phosphate binding protein (pbp) secretion signal peptide. In other embodiments the process can provide a protein recoverable, at least in part, externally to the cell. The process may also include the step of purifying the recombinant protein from the extracellular media. The recombinant polypeptide can include a signal targeting it to the extracellular environment, for example, a TPS secretion signal. The secretion or excretion signals can be expressed fused to the protein and the signal-linked protein can be purified from the media. Embodiments can thus include this isolated peptide as a fusion protein of the secretion signal and a protein or peptide of interest. However, the secretion signal can also be cleaved from the protein when the protein is targeted to the periplasm. In another embodiment, the linkage between the secretion signal and the protein or peptide can be modified to increase cleavage of the secretion signal.

In some embodiments, the process can produce a soluble recombinant protein. In other embodiments, the increase in level of the redox cofactor may produce active recombinant protein. The process of the invention may also lead to increased yield of recombinant protein as compared to when the protein is expressed without the redox cofactor because the protein folded in a native configuration will not be as prone to degradation in the cell.

Embodiments of the present invention include processes that produce at least 0.1 g/L soluble and/or active protein. These processes can produce 0.1 to 1 g/L soluble and/or active protein in the cell. In other embodiments, the total protein produced can be at least about 2.0 to about 50.0 g/L. In some of these embodiments, the amount of soluble and/or active protein produced is at least about 5%, about 10%, about 15%, about 20%, about 25%, or more of total recombinant protein produced.

The protein or peptide of interest can be a therapeutically useful protein or peptide. In embodiments of the present invention, the protein is a complex of multiple subunits linked by at least one disulfide bond. In some embodiments, the protein or peptide is at least 200 kD in molecular weight. In other embodiments, the protein or peptide is 20, 30, 40, 50, 60, 70, 80, 90, 100 or more amino acids in length. In other embodiments large proteins of several thousand kD are produced.

Embodiments of the present invention can also provide a media composition that can be used to produce high levels of properly folded or active recombinant proteins or peptides. The media includes a redox cofactor or cofactor precursor such as ubiquinone, menaquinone or heme chloride as a main ingredient.

Additional embodiments of the present include include a host cell that has been modified to increase expression of at least one redox cofactor. In other embodiments, the host cell has been modified to express a modified redox cofactor. The cell can be modified to increase ubiquinone expression through over expression of the ubiX, ubiD, ubiA, menA, or hemA genes or any of the other genes coding for the biosynthesis of these cofactors. These genes can also include global gene regulatory functions, such asfnr, arcA or hemA, that may be involved in the regulation of many genes or gene pathways responsible for the synthesis of redox cofactors (or their precursors) needed for effective disulfide bond formation.

### BRIEF DESCRIPTION OF THE FIGURES

FIG. 1 shows two pathways by which disulfide bond formation and disulfide bond isomerization are catalyzed; and

FIGS. 2A and 2B show expression of 7 recombinant disulfide bond-containing proteins in both E. coli and P. fluorescens. Cells were cultured in 96-well plates with shaking, micro-aerophilic conditions, wherein E=E. coli, P=P. fluorescens, S=soluble cell fraction, I=insoluble cell fraction.

### DETAILED DESCRIPTION OF THE INVENTION

The present invention will now be described more fully hereinafter with reference to the accompanying figures, in which some embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art. Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

The present invention provides compositions and processes for producing high levels of recombinant protein derived from a cell expression system containing disulfide bonds by increasing the amount of redox cofactor in the cell.

Proteins of Interest

The host cell can be designed to express a recombinant protein or peptide. These can be of any species and of any size. In other embodiments, the recombinant protein or peptide can be a therapeutically useful protein or peptide. These proteins may be monomeric or multimeric, and can contain any number of intra-chain or inter-chain disulfide bonds required for holo-protein stability and/or activity.

In some embodiments, the protein or peptide is a monomer. Other embodiments can include a protein or peptide that is a concatameric protein or peptide. When the protein is a concatamer, the separate units can be linked by a linker. In some embodiments, the linker is cleavable, for example, through a chemical treatment such as an acid treatment. In other embodiments, the units can be separated by a linker that allows purification of the concatamer, for example, as a tag sequence or a sequence with affinity for a purification agent.

The protein can, for example, be an antimicrobial protein or peptide. In some embodiments, antimicrobial peptides that are toxic to the host cell are produced. The invention is particularly useful for toxic agents because the agents are released from the cell and can be removed from the cell media, allowing enhanced growth and protein production.

In some embodiments, the protein can be a mammalian protein, such as a human protein, and can be, for example, a growth factor, a cytokine, a chemokine or a blood protein. The recombinant protein or peptide can be processed in a similar manner to the native protein or peptide. In some embodiments, the protein or peptide does not include a secretion signal in the coding sequence. In other embodiments, the recombinant protein or peptide is less than 100 kD, less than 50 kD, or less than 30 kD in size. In some embodiments, the recombinant protein or peptide is a peptide of at least 5, 10, 15, 20, 30, 40, 50 or 100 amino acids.

Extensive sequence information required for molecular genetics and genetic engineering techniques is publicly available. Access to complete nucleotide sequences of mammalian (including human) genes, cDNA sequences, amino acid sequences and genomes can be obtained from GenBank at the URL address http://www.ncbi.nlm.nih.gov/Entrez. Additional information can also be obtained from GeneCards, an electronic encyclopedia integrating information about genes and their products and biomedical applications from the Weizmann Institute of Science Genome and Bioinformatics (http://bioinformatics.weizmann.ac.il/cards/), nucleotide sequence information can be also obtained from the EMBL Nucleotide Sequence Database (http.//www.ebi.ac.uk/embl/) or the DNA Databank or Japan (DDBJ, http://www.ddbj.nig.ac.jp/; additional sites for information on amino acid sequences include Georgetown's protein information resource website (http://www-nbrf.georgetown.edu/pir/) and Swiss-Prot (http://au.expasy.org/sprot/sprot-top.html).

Examples of proteins that can be expressed in this invention include, but are not limited to, molecules, such as, for example: rennin; a growth hormone, including human growth hormone; bovine growth hormone; growth hormone releasing factor; parathyroid hormone; thyroid stimulating hormone; lipoproteins; α1-antitrypsin; insulin A-chain; insulin B-chain; proinsulin; thrombopoietin; follicle stimulating hormone; calcitonin; luteinizing hormone; glucagon; clotting factors, such as factor VIIIC, factor IX, tissue factor, and von Willebrands factor; anti-clotting factors, such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; a serum albumin, such as human serum albumin; mullerian-inhibiting substance; relaxin A-chain; relaxin B-chain; prorelaxin; mouse gonadotropin-associated peptide; a microbial protein, such as beta-lactamase; Dnase; inhibin; activin; vascular endothelial growth factor (VEGF); receptors for hormones or growth factors; integrin; protein A or D; rheumatoid factors; a neurotrophic factor, such as brain-derived neurotrophic factor (BDNF), neurotrophin-3, -4, -5, or -6 (NT-3, NT-4, NT-5, or NT-6), or a nerve growth factor, such as NGF-β; cardiotrophins (cardiac hypertrophy factor) such as cardiotrophin-1 (CT-1); platelet-derived growth factor (PDGF); fibroblast growth factor, such as aFGF and bFGF; epidermal growth factor (EGF); transforming growth factor (TGF), such as TGF-alpha and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins, such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), such as, M-CSF, GM-CSF, and G-CSF; interleukins (ILs), such as, IL-1 to IL-10; anti-HER-2 antibody; superoxide dismutase; T-cell receptors; surface membrane proteins; decay accelerating factor; viral antigen, such as, for example, a portion of the AIDS envelope; transport proteins; homing receptors; addressins; regulatory proteins; antibodies; and fragments of any of the above-listed polypeptides.

In some embodiments, the protein or peptide can be selected from IL-1, IL-1a, IL-1b, IL-2, IL-3, IL-4, IL-5, IL-6, IL-7, IL-8, IL-9, IL-10, IL-11, IL-12, IL-12elasti, IL-13, IL-15, IL-16, IL-18, IL-18BPa, IL-23, IL-24, VIP, erythropoietin, GM-CSF, G-CSF, M-CSF, platelet derived growth factor (PDGF), MSF, FLT-3 ligand, EGF, fibroblast growth factor (FGF; e.g., αFGF (FGF-1), βFGF (FGF-2), FGF-3, FGF-4, FGF-5, FGF-6, or FGF-7), insulin-like growth factors (e.g., IGF-1, IGF-2); tumor necrosis factors (e.g., TNF, Lymphotoxin), nerve growth factors (e.g., NGF), vascular endothelial growth factor (VEGF), interferons (e.g., IFN-α, IFN-β, IFN-γ), leukemia inhibitory factor (LIF), ciliary neurotrophic factor (CNTF), oncostatin M, stem cell factor (SCF), transforming growth factors (e.g., TGF-α, TGF-β1, TGF-β2, TGF-β3), TNF superfamily (e.g., LIGHT/TNFSF14, STALL-1/TNFSF13B (BLy5, BAFF, THANK), TNFalpha/TNFSF2 and TWEAK/TNFSF12), or chemokines (BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1/ABCD-1, MIP-1α, MIP-1β, MIP-2α/GROβ, MIP-3α/Exodus/LARC, MIP-3/Exodus-3/ELC, MIP-4/PARC/DC-CK1, PF-4, RANTES, SDF1, TARC, or TECK).

In one embodiment of the present invention, the production of recombinant multi-subunit proteins or peptides by a host cell of the species Pseudomonas is provided. Multisubunit proteins that can be expressed include homomeric and heteromeric proteins. The multisubunit proteins may include two or more subunits that may be the same or different. For example, the protein may be a homomeric protein comprising 2, 3, 4, 5, 6, 7, 8, 9, 10, 11, 12 or more subunits. The protein also may be a heteromeric protein including 2, 3, 4, 5, 6, 7, 8, 9, 10, 11, 12, or more subunits. Exemplary multisubunit proteins include: receptors including ion channel receptors; extracellular matrix proteins including chondroitin; collagen; immunomodulators including MHC proteins, full chain antibodies, and antibody fragments; enzymes including RNA polymerases, and DNA polymerases; and membrane proteins.

In another embodiment of the present invention, the production of blood proteins by a host cell can be provided. The blood proteins can include but are not limited to carrier proteins, such as albumin, including human and bovine albumin, transferrin, recombinant transferrin half-molecules, haptoglobin, fibrinogen and other coagulation factors, complement components, immunoglobulins, enzyme inhibitors, precursors of substances such as angiotensin and bradykinin, insulin, endothelin, and globulin, including alpha, beta, and gamma-globulin, and other types of proteins, peptides, and fragments thereof found primarily in the blood of mammals. The amino acid sequences for numerous blood proteins have been reported (see, S. S. Baldwin (1993) Comp. Biochem Physiol. 106b:203-218), including the amino acid sequence for human serum albumin (Lawn, L. M., et al. (1981) Nucleic Acids Research, 9:6103-6114) and human serum transferrin (Yang, F. et al. (1984) Proc. Natl. Acad. Sci. USA 81:2752-2756).

The production of recombinant enzymes or co-factors by a host cell of the species Pseudomonas fluorescens is provided in another embodiment of the present invention. The enzymes and co-factors expressed include, but are not limited to, aldolases, amine oxidases, amino acid oxidases, aspartases, B₁₂ dependent enzymes, carboxypeptidases, carboxyesterases, carboxylyases, chemotrypsin, CoA requiring enzymes, cyanohydrin synthetases, cystathione synthases, decarboxylases, dehydrogenases, alcohol dehydrogenases, dehydratases, diaphorases, dioxygenases, enoate reductases, epoxide hydrases, fumerases, galactose oxidases, glucose isomerases, glucose oxidases, glycosyltrasferases, methyltransferases, nitrile hydrases, nucleoside phosphorylases, oxidoreductases, oxynitilases, peptidases, glycosyltrasferases, peroxidases, enzymes fused to a therapeutically active polypeptide, tissue plasminogen activator, urokinase, reptilase, streptokinase, catalase, superoxide dismutase, Dnase, amino acid hydrolases (e.g., asparaginase, amidohydrolases), carboxypeptidases, proteases, trypsin, pepsin, chymotrypsin, papain, bromelain, collagenase, neuraminidase, lactase, maltase, sucrase, and arabinofuranosidases.

The production of recombinant single chain, Fab fragments and/or full chain antibodies or fragments or portions thereof by a host cell of the species Pseudomonas fluorescens is provided in another embodiment of the present invention. A single-chain antibody can include the antigen-binding regions of antibodies on a single stably-folded polypeptide chain. Fab fragments can be a piece of a particular antibody. The Fab fragment can contain the antigen binding site. The Fab fragment can contain two chains: a light chain and a heavy chain fragment. These fragments can be linked via a linker or a disulfide bond.

The coding sequence for the recombinant protein or peptide can be a native coding sequence for the target polypeptide. The coding sequence that can be optimized for use in the selected expression host cell: for example, by synthesizing the gene to reflect the codon use bias of a Pseudomonas species, such as P. fluorescens. The gene(s) that result can be constructed within or can be inserted into one or more vector, which can then be transformed into the expression host cell. Nucleic acids or polynucleotides provided in “expressible form” means nucleic acid or a polynucleotide that contains at least one gene that can be expressed by the selected bacterial expression host cell.

In some embodiments, the protein of interest is, or is substantially homologous to, a native protein, such as a native mammalian or human protein. In such embodiments, the protein is not found in a concatameric form, but is linked only to a secretion signal and optionally a tag sequence for purification and/or recognition.

In other embodiments, the protein of interest is a protein that is active at a temperature from about 20° C. to about 42° C. In other embodiments, the protein is active at physiological temperatures and is inactivated when heated to high or extreme temperatures, such as temperatures over 65° C.

In one embodiment, the protein of interest: is a protein that is active at a temperature from about 20° C. to about 42° C. and/or is inactivated when heated to high or extreme temperatures, such as temperatures over 65° C.; is, or is substantially homologous to, a native protein, such as a native mammalian or human protein and not expressed from nucleic acids in concatameric form; and the promoter is not a native promoter in P. fluorescens but is derived from another organism, such as E. coli.

Signal Sequences

In particular embodiments of the present invention, the disulfide bond containing proteins can include a signal sequence that increases expression of the protein in the periplasm of the cell. In other embodiments, the additional signal is targeted to the Sec secretion system. A signature of Sec-dependent protein export is the presence of a short (about 30 amino acids), mainly hydrophobic amino-terminal signal sequence in the exported protein. The signal sequence aids protein export and is cleaved off by a periplasmic signal peptidase when the exported protein reaches the periplasm. A typical N-terminal Sec-signal peptide contains an N-domain with at least one arginine or lysine residue, followed by a domain that contains a stretch of hydrophobic residues, and a C-domain containing the cleavage site for signal peptidases.

Signal peptides for the sec pathway can generally consist of the following three domains: (i) a positively charged n-region, (ii) a hydrophobic h-region and (iii) an uncharged polar c-region. The cleavage site for the signal peptidase is located in the c-region. However, the degree of signal sequence conservation and length, as well as the cleavage site position, can vary between different proteins. The secretion signal sequence can, for example, be any sequence that is identified by using a computer program designed to identify secretion signals, such as the SignalP program or as described in Hiller, et al. (2004) Nucleic Acids Research 32 (Web Server issue):W375-W379; available on the internet at URL: http://www.predisi.de.

In other embodiments, the protein, when produced, can also include an additional targeting sequence, such as a sequence that targets the protein to the extracellular medium. In some embodiments, the additional targeting sequence can be operably linked to the carboxy-terminus of the protein. In other embodiments, the protein can include a secretion signal for an autotransporter, a two partner secretion system, a main terminal branch system or a fimbrial usher porin. This additional sequence can, for example, be, or be substantially homologous to, a P. fluorescens Sec-system secretion peptide selected from a phosphate binding protein (pbp) secretion signal, an Outer Membrane Porin E (OprE) secretion signal, a Lys-Arg-Orn binding protein secretion signal, an azurin secretion signal, an iron (III) binding protein secretion signal and a lipoprotein B secretion signal. In some embodiments, the peptide sequence can be substantially homologous to the sequence of a phosphate binding protein (pbp) secretion signal peptide of at least amino acids: Met Lys Leu Lys Arg Leu Met Ala Ala Met Thr Phe Val Ala Ala Gly Val Ala Thr Ala Asn Ala Val Ala (SEQ ID NO: 1). In another particular embodiment, the secretion signal sequence is, or is substantially homologous to, the amino acid sequence: MPTTPHSFHLSPQGKLRWAIASLFLLPQLALA (SEQ ID NO:2).

In another embodiment, the secretion signal can be a secretion signal derived from an E. coli protein. The secretion signal can be a native signal sequence for an E. coli derived protein. The protein can be a chaperone protein. The protein can be a disulfide bond-forming protein. The sequence can be the native sequence of an E. coli chaperone protein such as a Skp protein.

Sequence Homology

As used herein, the term “homologous” can mean either: i) a protein or peptide that has an amino acid sequence that is substantially similar (i.e., at least 70%, 75%, 80%, 85%, 90%, 95%, 96%, 97%, 98% or 99%) to the sequence of a given original protein or peptide and that retains a desired function of the original protein or peptide; or ii) a nucleic acid that has a sequence that is substantially similar (i.e., at least 60%, 65%, 70%, 75%, 80%, 85%, 90%, 95%, 96%, 97%, 98% or 99%) to the sequence of a given nucleic acid and that retains a desired function of the original nucleic acid sequence. In all of the embodiments of this invention and disclosure, any disclosed protein, peptide or nucleic acid can be substituted with a homologous or substantially homologous protein, peptide, or nucleic acid that retains a desired function. In all of the embodiments of this invention and disclosure, when any nucleic acid is disclosed, it should be assumed that the invention also includes all nucleic acids that hybridize to the disclosed nucleic acid. This term can also mean “identity” or “similarity.” As known in the art, these terms define relationships between two polypeptide sequences or two polynucleotide sequences, as determined by comparing the sequences. In the art, identity also means the degree of sequence relatedness between two polypeptide or two polynucleotide sequences as determined by the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity or similarity between two sequences include, but are not limited to, those disclosed in Carillo, H., and Lipman, D., SIAM J. Applied Math., 48:1073 (1988). Preferred methods to determine identity are designed to give the largest match between the two sequences tested. Methods to determine identity and similarity are codified in computer programs. Typical computer program methods to determine identity and similarity between two sequences include GCG program package (Devereux, J., et al., Nucleic Acids Research 12(1):387 (1984)), BLASTP, BLASTN, FASTA and TFASTA (Atschul, S. F. et al., J. Mol. Biol. 215:403 (1990)).

In embodiments of the present invention, the amino acid sequence of the homologous polypeptide can be a variant of a given original polypeptide, wherein the sequence of the variant is obtainable by replacing up to or about 30% of the original polypeptide's amino acid residues with other amino acid residue(s), including 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30%, provided that the substituted variant retains a desired function of the original polypeptide. A variant amino acid with substantial homology will be at least about 70% homologous to the given polypeptide, or 70, 75, 80, 85, 90, 95, 98, 99 or 100% homologous.

In embodiments of the present invention, a variant can be similarly substituted or a similar variant of the original polypeptide. The term “similarly substituted variant” means a variant containing, relative to the original polypeptide, different residues that are “similar” amino acid residue substitutions, but in which not all differences are “similar” substitutions. As used herein, the term “similar variant” means a variant in which each of the different residues is a “similar” amino acid residue substitution. As used in this context, the term “similar” amino acid residue refers to those residues that are members of any one of the 15 conservative or semi-conservative groups shown in Table 1.

TABLE 1
Similar Amino Acid Substitution Groups

| Conservative Groups (8) | Semi-Conservative Groups (7) |
|---|---|
| Arg, Lys | Arg, Lys, His |
| Asp, Glu | Asn, Asp, Glu, Gln |
| Asn, Gln | |
| Ile, Leu, Val | Ile, Leu, Val, Met, Phe |
| Ala, Gly | Ala, Gly, Pro, Ser, Thr |
| Ser, Thr | Ser, Thr, Tyr |
| Phe, Tyr | Phe, Trp, Tyr |
| Cys (non-cystine), Ser | Cys (non-cystine), Ser, Thr |

In embodiments of the present invention, at least 50% of the substitutions will appear as conservative amino acid substitutions, and the remainder of the substitutions will be semi-conservative substitutions. In other embodiments, at least 60%, or at least 70%, or at least 75%, or at least 80%, or at least 85%, or at least 90%, or at least 95% of the similar substitutions will appear as conservative amino acid substitutions.

In embodiments of the present invention, at least 50% of the similar substitutions will appear as conservative amino acid substitutions, with the remainder of the similar substitutions appearing as semi-conservative substitutions. In other embodiments, at least 55%, or at least 60%, or at least 65%, or at least 70%, or at least 75%, or at least 80%, or at least 90% or at least 95% of the similar substitutions will appear as conservative amino acid substitutions. In one embodiment, a substituted variant will be a similar variant of a given original polypeptide.

Nucleic Acid Sequences

In embodiments of the present invention can also include nucleic acids that encode a disulfide bond containing protein that also include a second nucleic acid sequence that encodes a secretion signal peptide. The secretion signal peptide can be a signal targeted to the periplasm. In particular embodiments, the additional signal is targeted to the Sec secretion system. This additional sequence can, for example, be, or be substantially homologous to, a P. fluorescens Sec-system secretion peptide selected from a pbp, OprE, Lys-Arg-Om binding protein, azurin, iron (III) binding protein, a DsbC protein or a lipoprotein B secretion signal. In another particular embodiment, the secretion signal sequence is, or is substantially homologous to, the amino acid sequence: MPTTPHSFHLSPQGKLRWAIASLFLLPQLALA (SEQ ID NO:2). In one embodiment, the peptide sequence is that is, or is substantially homologous to, the sequence of a phosphate binding protein (pbp) secretion signal peptide of at least amino acids: Met Lys Leu Lys Arg Leu Met Ala Ala Met Thr Phe Val Ala Ala Gly Val Ala Thr Ala Asn Ala Val Ala (SEQ ID NO:1).

In other embodiments, the secretion signal is a secretion signal derived from an E. coli protein. In some embodiments, the secretion signal can be a native signal sequence for an E. coli derived protein. In certain embodiments, the protein can be a chaperone protein. The protein can be a disulfide bond-forming protein. In other embodiments, the sequence can be the native sequence of an E. coli chaperone protein, such as a skp protein.

In certain embodiments, the nucleic acid sequence of the nucleic acid is adjusted based on the codon usage of a host organism. Codon usage or codon preference is well known in the art. The selected coding sequence may be modified by altering the genetic code thereof to match that employed by the bacterial host cell, and the codon sequence thereof may be enhanced to better approximate that employed by the host. Genetic code selection and codon frequency enhancement may be performed according to any of the various methods known to one of ordinary skill in the art, e.g., oligonucleotide-directed mutagenesis. Useful on-line InterNet resources to assist in this process include, e.g.: (1) the Codon Usage Database of the Kazusa DNA Research Institute (2-6-7 Kazusa-kamatari, Kisarazu, Chiba 292-0818 Japan) and available at http://www.kazusa.or.jp/codon/; and (2) the Genetic Codes tables available from the NCBI Taxonomy database at http://www.ncbi.nlm.nih.gov/Taxonomy/Utils/wprintgc.cgi?mode=c. For example, Pseudomonas species are reported as utilizing Genetic Code Translation Table 11 of the NCBI Taxonomy site, and at the Kazusa site as exhibiting the codon usage frequency of the table shown at http://www.kazusa.or.jp/codon/cgibin/.

Nucleic Acid Homology

It is apparent to one of skill in the art that a variety of substantially homologous nucleic acids can be provided that encode sequences of substantially similar peptides. In the case of homology for coding sequences, a coding sequence homologous to a protein-encoding nucleic acid sequence hereof will contain no more than 30% (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30%) mutations that cause a change in reading frame and none that create a premature stop codon, as compared to the protein-encoding nucleic acid sequence disclosed herein. However, the nucleic acid sequences can be designed based on differing codon usage in the desired expression systems.

Nucleic Sequence homology is determined according to any of various methods well known in the art. Examples of useful sequence alignment and homology determination methodologies include those described below.

Alignments and searches for homologous sequences can be performed using the U.S. National Center for Biotechnology Information (NCBI) program, MegaBLAST (currently available at http://www.ncbi.nlm.nih.gov/BLAST/). Use of this program with options for percent identity set at 70% for amino acid sequences, or set at 90% for nucleotide sequences, will identify those sequences with 70%, or 90%, or greater homology to the query sequence. Other software known in the art is also available for aligning and/or searching for homologous sequences, e.g., sequences at least 70% or 90% homologous to an information string containing a promoter base sequence or activator-protein-encoding base sequence according to the present invention. For example, sequence alignments for comparison to identify sequences at least 70% or 90% homologous to a query sequence can be performed by use of, e.g., the GAP, BESTFIT, BLAST, FASTA, and TFASTA programs available in the GCG Sequence Analysis Software Package (available from the Genetics Computer Group, University of Wisconsin Biotechnology Center, 1710 University Avenue, Madison, Wis. 53705), with the default parameters as specified therein, plus a parameter for the extent of homology set at 70% or 90%. Also, for example, the CLUSTAL program (available in the PC/Gene software package from Intelligenetics, Mountain View, Calif.) may be used.

These and other sequence alignment methods are well known in the art and may be conducted by manual alignment, by visual inspection, or by manual or automatic application of a sequence alignment algorithm, such as any of those embodied by the above-described programs. Various useful algorithms include, e.g.: the similarity search method described in W. R. Pearson & D. J. Lipman, Proc. Nat'l Acad. Sci. USA 85:2444-48 (April 1988); the local homology method described in T. F. Smith & M. S. Waterman, in Adv. Appl. Math. 2:482-89 (1981) and in J. Molec. Biol. 147:195-97 (1981); the homology alignment method described in S. B. Needleman & C. D. Wunsch, J. Molec. Biol. 48(3):443-53 (March 1970); and the various methods described, e.g., by W. R. Pearson, in Genomics 11(3):635-50 (November 1991); by W. R. Pearson, in Methods Molec. Biol. 24:307-31 and 25:365-89 (1994); and by D. G. Higgins & P. M. Sharp, in Comp. Appl'ns in Biosci. 5:151-53 (1989) and in Gene 73(1):237-44 (15 Dec. 1988).

Nucleic acid hybridization performed under highly stringent hybridization conditions is also a useful technique for obtaining sufficiently homologous sequences for use herein. Highly stringent hybridization conditions generally means hybridization performed in aqueous conditions at at least 68° C.

Vectors

Expressible coding sequences will be operatively attached to a transcription promoter capable of functioning in the chosen host cell, as well as all other required transcription and translation regulatory elements. The term “operably attached” refers to any configuration in which the transcriptional and any translational regulatory elements are covalently attached to the encoding sequence in such disposition(s), relative to the coding sequence, that in and by action of the host cell, the regulatory elements can direct the expression of the coding sequence.

The vector will typically comprise one or more phenotypic selectable markers and an origin of replication to ensure maintenance of the vector and to, if desirable, provide amplification within the host. Suitable hosts for transformation in accordance with the present disclosure include various species within the genera Pseudomonas, and particularly preferred is the host cell strain of P. fluorescens.

In embodiments of the present invention, the vector can include a coding sequence for expression of a recombinant protein or peptide of interest. The recombinant proteins and peptides can be expressed from polynucleotides in which the target polypeptide coding sequence is operably attached to the leader sequence and transcription and translation regulatory elements to form a functional gene from which the host cell can express the protein or peptide. The coding sequence can be a native coding sequence for the target polypeptide, if available, but will more preferably be a coding sequence that has been selected, improved, or optimized for use in the selected expression host cell: for example, by synthesizing the gene to reflect the codon use bias of a host species. In certain embodiments of the invention, the host species is a P. fluorescens, and the codon bias of P. fluorescens is taken into account when designing both the signal sequence and the protein or peptide sequence. The gene(s) are constructed within or inserted into one or more vector(s), which can then be transformed into the expression host cell.

Other regulatory elements can be included in a vector (also termed “expression construct”). Such elements include, but are not limited to, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” peptide coding sequences, which facilitates identification, separation, purification, or isolation of an expressed polypeptide.

In other embodiments, the expression vector can further comprise a tag sequence adjacent to the coding sequence for the secretion signal or to the coding sequence for the recombinant protein or peptide. In certain embodiments, this tag sequence allows for purification of the protein. The tag sequence can be an affinity tag, such as a hexa-histidine affinity tag. In other embodiments, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification.

A protein-encoding gene according to the present invention can include, in addition to the protein coding sequence, the following regulatory elements operably linked thereto: a promoter, a ribosome binding site (RBS), a transcription terminator, translational start and stop signals. RBSs can be obtained from any of the species useful as host cells in expression systems according to the present invention, preferably from the selected host cell. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12): 1123-30 (December 2001). In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989) (native RBS sequence of AAGGAAG). Further examples of methods, vectors, and translation and transcription elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox.

Transcription of the DNA encoding the proteins of the present invention by Pseudomonas can be increased by inserting an enhancer sequence into the vector or plasmid. Typical enhancers are cis-acting elements of DNA, usually about from 10 to 300 bp in size that act on the promoter to increase its transcription. Examples include various Pseudomonas enhancers.

Generally, the recombinant expression vectors will include origins of replication and selectable markers permitting transformation of the Pseudomonas host cell and a promoter derived from a highly-expressed gene to direct transcription of a downstream structural sequence. Such promoters can be derived from operons encoding the enzymes such as 3-phosphoglycerate kinase (PGK), acid phosphatase, or heat shock proteins, among others. The heterologous structural sequence is assembled in appropriate phase with translation initiation and termination sequences, and preferably, a leader sequence capable of directing secretion of the translated enzyme. Optionally, the heterologous sequence can encode a fusion enzyme including an N-terminal identification peptide imparting desired characteristics, e.g., stabilization or simplified purification of expressed recombinant product.

Vectors are known in the art as useful for expressing recombinant proteins in host cells, and any of these may be used for expressing the genes according to the present invention. Such vectors include, e.g., plasmids, cosmids, and phage expression vectors. Examples of useful plasmid vectors include, but are not limited to, the expression plasmids pBBR1MCS, pDSK519, pKT240, pML122, pPS10, RK2, RK6, pRO1600, and RSF1010. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001).

Further examples of expression vectors that can be used in Pseudomonas host cells include those listed in Table 2 as derived from the indicated replicons.

TABLE 2
Examples of Expression Vectors

| Replicon | Vector(s) |
|---|---|
| PPS10 | PCN39, PCN51 |
| RSF1010 | PKT261-3 |
| | PMMB66EH |
| | PEB8 |
| | PPLGN1 |
| | PMYC1050 |
| RK2/RP1 | PRK415 |
| | PJB653 |
| PRO1600 | PUCP |
| | PBSP |

The expression plasmid, RSF1010, is described, e.g., by F. Heffron et al., in Proc. Nat'l Acad. Sci. USA 72(9):3623-27 (September 1975), and by K. Nagahari & K. Sakaguchi, in J. Bact. 133(3):1527-29 (March 1978). Plasmid RSF1010 and derivatives thereof are particularly useful vectors in the present invention. Exemplary, useful derivatives of RSF1010, which are known in the art, include, e.g., pKT212, pKT214, pKT231 and related plasmids, and pMYC1050 and related plasmids (see, e.g., U.S. Pat. Nos. 5,527,883 and 5,840,554 to Thompson et al.), such as, e.g., pMYC1803. Plasmid pMYC1803 is derived from the RSF1010-based plasmid pTJS260 (see U.S. Pat. No. 5,169,760 to Wilcox), which carries a regulated tetracycline resistance marker and the replication and mobilization loci from the RSF1010 plasmid. Other exemplary useful vectors include those described in U.S. Pat. No. 4,680,264 to Puhler et al.

In one embodiment, an expression plasmid is used as the expression vector. In another embodiment, RSF1010 or a derivative thereof is used as the expression vector. In still another embodiment, pMYC1050 or a derivative thereof, or pMYC1803 or a derivative thereof, is used as the expression vector.

The plasmid can be maintained in the host cell by use of a selection marker gene, also present in the plasmid. This may be an antibiotic resistance gene(s), in which case the corresponding antibiotic(s) will be added to the fermentation medium, or any other type of selection marker gene known as useful in the art, e.g., a prototrophy-restoring gene in which case the plasmid will be used in a host cell that is auxotrophic for the corresponding trait, e.g., a biocatalytic trait such as an amino acid biosynthesis or a nucleotide biosynthesis trait or a carbon source utilization trait.

The promoters used in accordance with the present invention can be constitutive promoters or regulated promoters. Common examples of useful regulated promoters include those of the family derived from the lac promoter (i.e., the lacZ promoter), especially the tac and trc promoters described in U.S. Pat. No. 4,551,433 to DeBoer, as well as Ptac16, Ptac17, PtacII, PlacUV5, and the T7lac promoter. In certain embodiments of the present invention the promoter is not derived from the host cell organism. In some embodiments, the promoter is derived from an E. coli organism.

Examples of non-lac-type promoters that are used in expression systems according to embodiments of the present invention include, e.g., those listed in Table 3.

TABLE 3
Examples of non-lac Promoters

| Promoter | Inducer |
|---|---|
| P_{R} | High temperature |
| P_{L} | High temperature |
| Pm | Alkyl- or halo-benzoates |
| Pu | Alkyl- or halo-toluenes |
| Psal | Salicylates |

See, e.g.: J. Sanchez-Romero & V. De Lorenzo (1999) Genetic Engineering of Nonpathogenic Pseudomonas strains as Biocatalysts for Industrial and Environmental Processes, in Manual of Industrial Microbiology and Biotechnology (A. Demain & J. Davies, eds.) pp. 460-74 (ASM Press, Washington, D.C.); H. Schweizer (2001) Vectors to express foreign genes and techniques to monitor gene expression for Pseudomonads, Current Opinion in Biotechnology, 12:439-445; and R. Slater & R. Williams (2000) The Expression of Foreign DNA in Bacteria, in Molecular Biology and Biotechnology (J. Walker & R. Rapley, eds.) pp. 125-54 (The Royal Society of Chemistry, Cambridge, UK)). A promoter having the nucleotide sequence of a promoter native to the selected bacterial host cell can also be used to control expression of the transgene encoding the target polypeptide, e.g, a Pseudomonas anthranilate or benzoate operon promoter (Pant, Pben). Tandem promoters can also be used in which more than one promoter is covalently attached to another, whether the same or different in sequence, e.g., a Pant-Pben tandem promoter (interpromoter hybrid) or a Plac-Plac tandem promoter.

Regulated promoters utilize promoter regulatory proteins in order to control transcription of the gene of which the promoter is a part. Where a regulated promoter is used herein, a corresponding promoter regulatory protein will also be part of an expression system according to the present invention. Examples of promoter regulatory proteins include, but are not limited to, activator proteins, e.g., E. coli catabolite activator protein, MalT protein; AraC family transcriptional activators; repressor proteins, e.g., E. coli LacI proteins; and dual-fuction regulatory proteins, e.g., E. coli NagC protein. Many regulated-promoter/ promoter-regulatory-protein pairs are known in the art.

Promoter regulatory proteins interact with an effector compound, i.e., a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter, promoter-regulatory-protein, effector-compound trios are known in the art. Although an effector compound can be used throughout the cell culture or fermentation, in a preferred embodiment in which a regulated promoter is used, after growth of a desired quantity or density of host cell biomass, an appropriate effector compound is added to the culture in order to directly or indirectly result in expression of the desired target gene(s).

By way of example, where a lac family promoter is utilized, a lacI gene can also be present in the system. The lacI gene, which is (normally) a constitutively expressed gene, encodes the Lac repressor protein (LacI protein) which binds to the lac operator of these promoters. Thus, where a lac family promoter is utilized, the lacI gene can also be included and expressed in the expression system. In the case of the lac promoter family members, e.g., the tac promoter, the effector compound is an inducer, preferably a gratuitous inducer such as IPTG (isopropyl-D-1-thiogalactopyranoside, also called “isopropylthiogalactoside”).

The Champion™ pET expression system provides a high level of protein production. Expression is induced from the strong T7lac promoter. This system takes advantage of the high activity and specificity of the bacteriophage T7 RNA polymerase for high level transcription of the gene of interest. The lac operator located in the promoter region provides tighter regulation than traditional T7-based vectors, improving plasmid stability and cell viability (Studier, F. W. and B. A. Moffatt (1986) J Molecular Biology 189(1):113-30; Rosenberg, et al. (1987) Gene 56(1):125-35). The T7 expression system uses the T7 promoter and T7 RNA polymerase (T7 RNAP) for high-level transcription of the gene of interest. High-level expression is achieved in T7 expression systems because the T7 RNAP is more processive than native E. coli RNAP and is dedicated to the transcription of the gene of interest. Expression of the identified gene is induced by providing a source of T7 RNAP in the host cell. This is accomplished by using a BL21 E. coli host containing a chromosomal copy of the T7 RNAP gene. The T7 RNAP gene is under the control of the lacUV5 promoter which can be induced by IPTG. T7 RNAP is expressed upon induction and transcribes the gene of interest.

The pBAD expression system allows tightly controlled, titratable expression of recombinant protein through the presence of specific carbon sources such as glucose, glycerol and arabinose (Guzman, et al. (1995) J Bacteriology 177(14):4121-30). The pBAD vectors are uniquely designed to give precise control over expression levels. Heterologous gene expression from the pBAD vectors is initiated at the araBAD promoter. The promoter is both positively and negatively regulated by the product of the araC gene. AraC is a transcriptional regulator that forms a complex with L-arabinose. In the absence of L-arabinose, the AraC dimer blocks transcription. For maximum transcriptional activation two events are required: (i.) L-arabinose binds to AraC allowing transcription to begin; and (ii.) the cAMP activator protein (CAP)-cAMP complex binds to the DNA and stimulates binding of AraC to the correct location of the promoter region.

The trc expression system allows high-level, regulated expression in E. coli from the trc promoter. The trc expression vectors have been optimized for expression of eukaryotic genes in E. coli. The trc promoter is a strong hybrid promoter derived from the tryptophane (trp) and lactose (lac) promoters. It is regulated by the lacO operator and the product of the lacIQ gene (Brosius, J. (1984) Gene 27(2):161-72).

Expression Systems

In embodiments of the present invention, an improved expression system for the production of recombinant protein is provided. In certain embodiments the system can include a host cell that has increased levels of a redox cofactor and increased levels of oxido-reductase/isomerase enzymes. In certain embodiments of the present invention, the increased levels of a redox cofactor and increased levels of oxido-reductase/isomerase enzymes are in the periplasm of the host cell, and can be confined to the periplasm.

In another embodiment of the present invention, the host cell has been genetically modified to increase levels of redox cofactor by increasing the expression of a gene or genes that is (are) involved in cofactor biosynthesis. In other embodiments the cell has been modified to increase uptake of a cofactor, or of a precursor of a cofactor, from the environment. In certain embodiments, the cell has been modified to increase the permeability of the extracellular membrane. The modification can be by genetic manipulation, or can be by pharmacological means. For example, the cell can be modified by altering the permeability of the membrane by incubation at different concentrations of salt. The cell can also be made porous by incubation with certain polymers.

The secretion system can also include a fermentation media that increases the levels of redox cofactor in the cell. In embodiments of the present invention, the level of cofactor can be increased by including the cofactor in the media. In some embodiments the system includes a mineral salts media. In other embodiments, the system includes a chemical inducer in the media.

The system can also include a promoter, which can be a selectable promoter, and an inducer. In some cases, this promoter is a promoter not native to P. fluorescens, such as an E. coli promoter. In some embodiments this promoter is, for example, an inducible promoter such as a lac promoter. The promoter can also be a hybrid of several different promoters, at least one of which is not native to a P. fluorescens organism. For example, the promoter can be a trc promoter. The promoter can also, for example, be a tac promoter.

The cells of the system can also be otherwise modified to increase production of recombinant protein. In embodiments of the present invention the cell can be altered to reduce the activity of a protease. In some embodiments, the protease is present in the periplasmic compartment. In certain embodiments, the protease is a periplasmic protease and is DegP. In other embodiments the protease is an extracellular protease and can be SphB1. In yet other embodiments, the protease can be an alkaline metalloprotease, and in a particular subembodiment, is AprA.

Process

A process and system for preparing a recombinant protein in a host cell is provided. Embodiments of the present invention include increasing the level of at least one redox cofactor in the host cell. In certain embodiments of the present invention, the process includes adding a redox cofactor into the cell media. In a subembodiment of this process, the cell membrane is permeabilized to increase uptake of the redox cofactor. In separate embodiments, the level of redox cofactor is increased in the cell. The level can be changed by inducing expression of a redox cofactor in the cell from a recombinant DNA. The level can also be changed by altering the growth characteristics of the cell.

In embodiments of the present invention, the expression can occur at high density cell culture. In certain embodiments, the level of redox cofactor can be altered by changing the growth conditions of the cell. This could be accomplished by growing the cells in the presence of low level, sub-lethal amounts of an agent that would create oxidative stress, i.e., as in the presence of reducing agents such as dithiothreitol or thioglycerol. Also, growing cells at high density in a strongly oxidative environment through vigorous aeration or the addition to the medium of oxidative factors like hydrogen peroxide or copper chloride could have a similar effect. Alternatively, if overexpression of genes coding the protein disulfide bond isomerase/oxido-reductase enzymes or pathway of isomerases and accessory proteins does not lead to more efficient folding and disulfide bond formation in the heterologous protein, it may be due to the availability of the redox cofactor becoming rate limiting to the reaction. Therefore, overexpression of the isomerases/oxido-reductases only leads to the production of inactive protein. It may be necessary not only to over-express the isomerases, but also derepress or un-regulate to some extent the pathway(s) that supply the redox cofactors to the isomerases at the same time. Doing both may lead to an enhancement of the folding and disulfide bond formation pathways in microbes like Escherichia coli or Pseudomonas fluorescens. This enhancement would lead to the ability to recover more active heterologous protein from these organisms and reduce the cost of their production.

In embodiments of the present invention, the process can provide for a protein in which the three dimensional structure is consistent with the native protein. The process may also include the step of purifying the recombinant protein from the cell or from the extracellular media. In some embodiments, the recombinant protein is purified from the host cell periplasm. In other embodiments, the recombinant protein can be purified from inclusion bodies in the host cell cytoplasm or periplasm. In yet other embodiments, the recombinant protein is purified from the extracellular environment.

In embodiments of the present invention, the process can also produce protein localized to the periplasm of the host cell. In certain embodiments, the process can produce properly processed recombinant protein in the extracellular space. In other embodiments, the expression of the secretion peptide can produce active recombinant protein in the extracellular space. The process of the invention can also lead to increased yield of native, active recombinant protein as compared to when the protein is expressed without the addition of redox cofactors.

In certain embodiments of the present invetion, the process produces at least 0.1 g/L of disulfide bonded protein. In other embodiments, the process produces 0.1 to 10 g/L of disulfide bonded protein. In subembodiments, the process produces at least about 0.2, 0.3, 0.4, 0.5, 0.6, 0.7, 0.8, 0.9 or 1.0 g/L of disulfide bonded protein. In some embodiments, the process produces more than 1.0 g/L, and can produce 2, 3, 4, 5, 6, 7, 8, 9, 10 or more g/L of disulfide bonded protein. In other embodiments, the process produces 10 to 50 g/L of disulfide bonded protein. In some embodiments, the amount of disulfide bonded protein produced is at least about 5%, about 10%, about 15%, about 20%, about 25%, about 30%, about 40%, about 50%, about 60%, about 70%, about 80%, about 90%, about 95% or more of total recombinant protein produced.

In embodiments of the present invention, the process can produce at least 0.1 g/L correctly processed protein. A correctly processed protein has an amino terminus of the native protein. In other embodiments, the process can produce 0.1 to 10 g/L correctly processed protein in the cell. In subembodiments, the process can produce at least about 0.2, 0.3, 0.4, 0.5, 0.6, 0.7, 0.8, 0.9 or 1.0 g/L correctly processed protein. In other embodiments, the total correctly processed recombinant protein produced is at least 1.0 g/L. In subembodiments, the total correctly processed protein produced can be at least about 2.0, 3.0, 4.0, 5.0, 6.0, 7.0, 8.0, 9.0, 10.0, 15.0, 20.0 or 50.0 g/L. In some embodiments, the amount of correctly processed protein produced is at least about 5%, about 10%, about 15%, about 20%, about 25%, about 30%, about 40%, about 50%, about 60%, about 70%, about 80%, about 90%, about 95% or more of total recombinant protein in a correctly processed form.

Embodiments of the present invention can include producing the protein in an active form. The term “active” means the presence of biological function or biological effect, wherein the biological function or effect is comparative or substantially corresponds to the biological function or effect of a corresponding native protein or peptide. In the context of proteins, this typically means that a polynucleotide or polypeptide comprises a biological function or effect that has at least about 20%, about 50%, at least about 60-80%, or at least about 90-95% activity compared to the corresponding native protein or peptide using standard parameters. The determination of protein or peptide activity can be performed utilizing corresponding standard, targeted comparative biological assays for particular proteins or peptides. One indication that a recombinant protein or peptide biological function or effect is that the recombinant polypeptide can be immunologically cross reactive with the native polypeptide.

In other embodiments of the present invention, more than 50% of the expressed, transgenic peptide, polypeptide, protein, or fragment thereof produced can be produced in a renaturable form in the cell or extracellular space. In another embodiment, about 60%, 70%, 75%, 80%, 85%, 90%, 95%, 96%, 97%, 98% or 99% of the expressed protein is obtained in or can be renatured into active form.

The process of the invention can also lead to increased yield of recombinant protein. In one embodiment, the process produces recombinant protein as 5, 10, 15, 20, 25, 30, 40 or 50, 55, 60, 65, 70, or 75% of total cell protein (tcp). “Percent total cell protein” is the amount of protein or peptide in the host cell as a percentage of aggregate cellular protein. The determination of the percent total cell protein is well known in the art.

In particular embodiments of the present invention, the host cell can have a recombinant peptide, polypeptide, protein, or fragment thereof expression level of at least 1% tcp and a cell density of at least 40 g/L, when grown (i.e., within a temperature range of about 4° C. to about 55° C., inclusive) in a mineral salts medium. In certain embodiments, the expression system can have a recombinant protein of peptide expression level of at least 5% tcp and a cell density of at least 40 g/L, when grown (i.e., within a temperature range of about 4° C. to about 55° C., inclusive) in a mineral salts medium at a fermentation scale of at least 10 Liters.

Host Cell

Certain embodiments of the present invention provide a P. fluorescens expression system for expression of recombinant protein. In these embodiments, the host cell can be selected from “Gram-negative Proteobacteria Subgroup 18.” “Gram-negative Proteobacteria Subgroup 18” is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): Pseudomonas fluorescens biotype A, also called biovar 1 or biovar I (ATCC 13525); Pseudomonas fluorescens biotype B, also called biovar 2 or biovar II (ATCC 17816); Pseudomonas fluorescens biotype C, also called biovar 3 or biovar III (ATCC 17400); Pseudomonas fluorescens biotype F, also called biovar 4 or biovar IV (ATCC 12983); Pseudomonas fluorescens biotype G, also called biovar 5 or biovar V (ATCC 17518); Pseudomonas fluorescens biovar VI; Pseudomonas fluorescens PfO-1; Pseudomonas fluorescens Pf-5 (ATCC BAA-477); Pseudomonas fluorescens SBW25; and Pseudomonas fluorescens subsp. cellulosa (NCIMB 10462). The host cell can also be selected from “Gram-negative Proteobacteria Subgroup 19.” “Gram-negative Proteobacteria Subgroup 19” is defined as the group of all strains of Pseudomonas fluorescens biotype A. A particularly preferred strain of this biotype is P. fluorescens strain MB101 (see U.S. Pat. No. 5,169,760 to Wilcox), and derivatives thereof. An example of a preferred derivative thereof is P. fluorescens strain MB214, constructed by inserting into the MB101 chromosomal asd (aspartate dehydrogenase gene) locus, a native E. coli PlacI-lacI-lacZYA construct (i.e., in which PlacZ was deleted).

Additional P. fluorescens strains that can be used in the present invention include Pseudomonas fluorescens Migula and Pseudomonas fluorescens Loitokitok, having the following ATCC designations: [NCIB 8286]; NRRL B-1244; NCIB 8865 strain CO1; NCIB 8866 strain CO2; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [IEM 15/47]; IAM 1008 [AHH-27]; IAM 1055 [AHH-23]; 1 [IFO 15842]; 12 [ATCC 25323; NIH 11; den Dooren de Jong 216]; 18 [IFO 15833; WRRL P-7]; 93 [TR-10]; 108 [52-22; IFO 15832]; 143 [IFO 15836; PL]; 149 [2-40-40; IFO 15838]; 182 [IFO 3081; PJ 73]; 184 [IFO 15830]; 185 [W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198 [PJ 302]; 201 [PJ 368]; 202 [PJ 372]; 203 [PJ 376]; 204 [IFO 15835; PJ 682]; 205 [PJ 686]; 206 [PJ 692]; 207 [PJ 693]; 208 [PJ 722]; 212 [PJ 832]; 215 [PJ 849]; 216 [PJ 885]; 267 [B-9]; 271 [B-1612]; 401 [C71A; IFO 15831; PJ 187]; NRRL B-3178 [4; IFO 15841]; KY 8521; 3081; 30-21; [IFO 3081]; N; PYR; PW; D946-B83 [BU 2183; FERM-P 3328]; P-2563 [FERM-P 2894; IFO 13658]; IAM-1126 [43F]; M-1; A506 [A5-06]; A505 [A5-05-1]; A526 [A5-26]; B69; 72; NRRL B-4290; PMW6 [NCIB 11615]; SC 12936; A1 [IFO 15839]; F 1847 [CDC-EB]; F 1848 [CDC 93]; NCIB 10586; P17; F-12; AmMS 257; PRA25; 6133D02; 6519E01; N1; SC15208; BNL-WVC; NCTC 2583 [NCIB 8194]; H13; 1013 [ATCC 11251; CCEB 295]; IFO 3903; 1062; or Pf-5.

Embodiments of the present invention can include a process for providing the expression of disulfide bond containing proteins with a P. fluorescens secretion peptide. In certain embodiments, the host cell can be any cell capable of producing recombinant protein or peptide, including a P. fluorescens cell, as described above. The most commonly used systems to produce recombinant proteins or peptides include certain bacterial cells, particularly E. coli, because of their relatively inexpensive growth requirements and potential capacity to produce protein in large batch cultures. Yeast is also used to express biologically relevant proteins and peptides, particularly for research purposes. Systems include Saccharomyces cerevisiae or Pichia pastoris. These systems are well characterized, provide generally acceptable levels of total protein expression and are comparatively fast and inexpensive. Insect cell expression systems have also emerged as an alternative for expressing recombinant proteins in biologically active form. In some cases, correctly folded proteins that are post-translationally modified can be produced. Mammalian cell expression systems, such as Chinese hamster ovary cells, have also been used for the expression of recombinant proteins. On a small scale, these expression systems are often effective. Certain biologics can be derived from proteins, particularly in animal or human health applications. In another embodiment, the host cell is a plant cell, including, but not limited to, a tobacco cell, corn, a cell from an Arabidopsis species, potato or rice cell. In other embodiments a multicellular organism can be analyzed or modified in the process to a transgenic organism. Techniques for analyzing and/or modifying a multicellular organism are generally based on techniques described for modifying cells described below.

In some embodiments, the host cell can be a prokaryote, such as a bacterial cell including, but not limited to, an Escherichia or a Pseudomonas species. Bacterial cells are described, for example, in “Biological Diversity: Bacteria and Archaeans,” a chapter of the On-Line Biology Book, provided by Dr M J Farabee of the Estrella Mountain Community College, Arizona, USA at URL: http://www.emc.maricopa.edu/faculty/farabee/ BIOBK/BioBookDiversity_{—2}.html. In some embodiments, the host cell can be a Pseudomonad cell, and can typically be a P. fluorescens cell. In other embodiments, the host cell can also be an E. coli cell. In other embodiments, the host cell can be a eukaryotic cell, for example an insect cell, including but not limited to a cell from a Spodoptera, Trichoplusia Drosophila or an Estigmene species, or a mammalian cell, including but not limited to a murine cell, a hamster cell, a monkey, a primate or a human cell.

In certain embodiments of the present invention, the host cell can be a member of any of the bacterial taxa. The cell can, for example, be a member of any species of eubacteria. The host can be a member any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In certain embodiments of a eubacterial host cell, the cell can be a member of any species of eubacteria, excluding Cyanobacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In some embodiments of the present invention, the host can be a Gamma Proteobacterial host, and the host can be member of any one of the taxa Aeromonadales, Alteromonadales, Enterobacteriales, Pseudomonadales, or Xanthomonadales; or a member of any species of the Enterobacteriales or Pseudomonadales. In certain embodiments of the present invention, the host cell can be of the order Enterobacteriales, the host cell will be a member of the family Enterobacteriaceae, or a member of any one of the genera Erwinia, Escherichia, or Serratia; or a member of the genus Escherichia. In other embodiments of a host cell of the order Pseudomonadales, the host cell will be a member of the family Pseudomonadaceae, even of the genus Pseudomonas. Gamma Proteobacterial hosts can include members of the species Escherichia coli and members of the species Pseudomonas fluorescens.

Other Pseudomonas organisms can also be useful. Pseudomonads and closely related species include Gram-negative Proteobacteria Subgroup 1, which include the group of Proteobacteria belonging to the families and/or genera described as “Gram-Negative Aerobic Rods and Cocci” by R. E. Buchanan and N. E. Gibbons (eds.), Bergey's Manual of Detenninative Bacteriology, pp. 217-289 (8th ed., 1974) (The Williams & Wilkins Co., Baltimore, Md., USA) (hereinafter “Bergey (1974)”). Table 4 presents these families and genera of organisms.

TABLE 4
Families and Genera Listed in the Part, “Gram-Negative
Aerobic Rods and Cocci” (in Bergey (1974))

| | |
|---|---|
| Family I. Pseudomonadaceae | Gluconobacter |
| | Pseudomonas |
| | Xanthomonas |
| | Zoogloea |
| Family II. Azotobacteraceae | Azomonas |
| | Azotobacter |
| | Beijerinckia |
| | Derxia |
| Family III. Rhizobiaceae | Agrobacterium |
| | Rhizobium |
| Family IV. Methylomonadaceae | Methylococcus |
| | Methylomonas |
| Family V. Halobacteriaceae | Halobacterium |
| | Halococcus |
| Other Genera | Acetobacter |
| | Alcaligenes |
| | Bordetella |
| | Brucella |
| | Francisella |
| | Thermus |

“Gram-negative Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition hosts can include cells from the genus Pseudomonas, Pseudomonas enalia (ATCC 14393), Pseudomonas nigrifaciens (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram-negative Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram-negative Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

Embodiments of the present invention include a host cell selected from “Gram-negative Proteobacteria Subgroup 2.” “Gram-negative Proteobacteria Subgroup 2” is defined as the group of Proteobacteria of the following genera (with the total numbers of catalog-listed, publicly-available, deposited strains thereof indicated in parenthesis, all deposited at ATCC, except as otherwise indicated): Acidomonas (2); Acetobacter (93); Gluconobacter (37); Brevundimonas (23); Beijerinckia (13); Derxia (2); Brucella (4); Agrobacterium (79); Chelatobacter (2); Ensifer (3); Rhizobium (144); Sinorhizobium (24); Blastomonas (1); Sphingomonas (27); Alcaligenes (88); Bordetella (43); Burkholderia (73); Ralstonia (33); Acidovorax (20); Hydrogenophaga (9); Zoogloea (9); Methylobacter (2); Methylocaldum (1 at NCIMB); Methylococcus (2); Methylomicrobium (2); Methylomonas (9); Methylosarcina (1); Methylosphaera; Azomonas (9); Azorhizophilus (5); Azotobacter (64); Cellvibrio (3); Oligella (5); Pseudomonas (1139); Francisella (4); Xanthomonas (229); Stenotrophomonas (50); and Oceanimonas (4).

Exemplary host cell species of “Gram-negative Proteobacteria Subgroup 2” include, but are not limited to, the following bacteria (with the ATCC or other deposit numbers of exemplary strain(s) thereof shown in parenthesis): Acidomonas methanolica (ATCC 43581); Acetobacter aceti (ATCC 15973); Gluconobacter oxydans (ATCC 19357); Brevundimonas diminuta (ATCC 11568); Beijerinckia indica (ATCC 9039 and ATCC 19361); Derxia gummosa (ATCC 15994); Brucella melitensis (ATCC 23456), Brucella abortus (ATCC 23448); Agrobacterium tumefaciens (ATCC 23308), Agrobacterium radiobacter (ATCC 19358), Agrobacterium rhizogenes (ATCC 11325); Chelatobacter heintzii (ATCC 29600); Ensifer adhaerens (ATCC 33212); Rhizobium leguminosarum (ATCC 10004); Sinorhizobium fredii (ATCC 35423); Blastomonas natatoria (ATCC 35951); Sphingomonas paucimobilis (ATCC 29837); Alcaligenes faecalis (ATCC 8750); Bordetella pertussis (ATCC 9797); Burkholderia cepacia (ATCC 25416); Ralstonia pickettii (ATCC 27511); Acidovoraxfacilis (ATCC 11228); Hydrogenophaga flava (ATCC 33667); Zoogloea ramigera (ATCC 19544); Methylobacter luteus (ATCC 49878); Methylocaldum gracile (NCIMB 11912); Methylococcus capsulatus (ATCC 19069); Methylomicrobium agile (ATCC 35068); Methylomonas methanica (ATCC 35067); Methylosarcina fibrata (ATCC 700909); Methylosphaera hansonii (ACAM 549); Azomonas agilis (ATCC 7494); Azorhizophilus paspali (ATCC 23833); Azotobacter chroococcum (ATCC 9043); Cellvibrio mixtus (UQM 2601); Oligella urethralis (ATCC 17960); Pseudomonas aeruginosa (ATCC 10145), Pseudomonas fluorescens (ATCC 35858); Francisella tularensis (ATCC 6223); Stenotrophomonas maltophilia (ATCC 13637); Xanthomonas campestris (ATCC 33913); and Oceanimonas doudoroffii (ATCC 27123).

Embodiments of the present invention include a host cell selected from “Gram-negative Proteobacteria Subgroup 3.” “Gram-negative Proteobacteria Subgroup 3” is defined as the group of Proteobacteria of the following genera: Brevundimonas; Agrobacterium; Rhizobium; Sinorhizobium; Blastomonas; Sphingomonas; Alcaligenes; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

Embodiments of the present invention include a host cell selected from “Gram-negative Proteobacteria Subgroup 4.” “Gram-negative Proteobacteria Subgroup 4” is defined as the group of Proteobacteria of the following genera: Brevundimonas; Blastomonas; Sphingomonas ; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

Embodiments of the present invention include a host cell selected from “Gram-negative Proteobacteria Subgroup 5.” “Gram-negative Proteobacteria Subgroup 5” is defined as the group of Proteobacteria of the following genera: Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

The host cell can be selected from “Gram-negative Proteobacteria Subgroup 6.” “Gram-negative Proteobacteria Subgroup 6” is defined as the group of Proteobacteria of the following genera: Brevundimonas; Blastomonas; Sphingomonas; Burkholderia; Ralstonia; Acidovorax; Hydrogenophaga; Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 7.” “Gram-negative Proteobacteria Subgroup 7” is defined as the group of Proteobacteria of the following genera: Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 8.” “Gram-negative Proteobacteria Subgroup 8” is defined as the group of Proteobacteria of the following genera: Brevundimonas; Blastomonas; Sphingomonas; Burkholderia; Ralstonia; Acidovorax; Hydrogenophaga; Pseudomonas; Stenotrophomonas; Xanthomonas; and Oceanimonas.

The host cell can be selected from “Gram-negative Proteobacteria Subgroup 9.” “Gram-negative Proteobacteria Subgroup 9” is defined as the group of Proteobacteria of the following genera: Brevundimonas; Burkholderia; Ralstonia; Acidovorax; Hydrogenophaga; Pseudomonas; Stenotrophomonas; and Oceanimonas.

The host cell can be selected from “Gram-negative Proteobacteria Subgroup 10.” “Gram-negative Proteobacteria Subgroup 10” is defined as the group of Proteobacteria of the following genera: Burkholderia; Ralstonia; Pseudomonas; Stenotrophomonas; and Xanthomonas.

The host cell can be selected from “Gram-negative Proteobacteria Subgroup 11.” “Gram-negative Proteobacteria Subgroup 11” is defined as the group of Proteobacteria of the genera: Pseudomonas; Stenotrophomonas; and Xanthomonas. The host cell can be selected from “Gram-negative Proteobacteria Subgroup 12.” “Gram-negative Proteobacteria Subgroup 12” is defined as the group of Proteobacteria of the following genera: Burkholderia; Ralstonia; Pseudomonas. The host cell can be selected from “Gram-negative Proteobacteria Subgroup 13.” “Gram-negative Proteobacteria Subgroup 13” is defined as the group of Proteobacteria of the following genera: Burkholderia; Ralstonia; Pseudomonas; and Xanthomonas. The host cell can be selected from “Gram-negative Proteobacteria Subgroup 14.” “Gram-negative Proteobacteria Subgroup 14” is defined as the group of Proteobacteria of the following genera: Pseudomonas and Xanthomonas. The host cell can be selected from “Gram-negative Proteobacteria Subgroup 15.” “Gram-negative Proteobacteria Subgroup 15” is defined as the group of Proteobacteria of the genus Pseudomonas.

The host cell can be selected from “Gram-negative Proteobacteria Subgroup 16.” “Gram-negative Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beijerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginata (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas fulva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis.

The host cell can be selected from “Gram-negative Proteobacteria Subgroup 17.” “Gram-negative Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

Other suitable hosts include those classified in other parts of the reference, such as Gram (+) Proteobacteria. In one embodiment, the host cell is an E. coli. The genome sequence for E. coli has been established for E. coli MG1655 (Blattner, et al. (1997) The complete genome sequence of Escherichia coli K-12 Science 277(5331):1453-74) and DNA microarrays are available commercially for E. coli K12 (MWG Inc, High Point, N.C.). E. coli can be cultured in either a rich medium such as Luria-Bertani (LB) (10 g/L tryptone, 5 g/L NaCl, 5 g/L yeast extract) or a defined minimal medium such as M9 (6 g/L Na₂HPO₄,3 g/L KH₂PO₄, 1 g/L NH₄Cl, 0.5 g/L NaCl, pH 7.4) with an appropriate carbon source such as 1% glucose. Routinely, an over night culture of E. coli cells is diluted and inoculated into fresh rich or minimal medium in either a shake flask or a fermentor and grown at 37° C. A host can also be of mammalian origin, such as a cell derived from a mammal including any human or non-human mammal. Mammals can include, but are not limited to primates, monkeys, porcine, ovine, bovine, rodents, ungulates, pigs, swine, sheep, lambs, goats, cattle, deer, mules, horses, monkeys, apes, dogs, cats, rats, and mice.

A host cell may also be of plant origin. Any plant can be selected for the identification of genes and regulatory sequences. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radiscchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini. In some embodiments, plants useful in the process are Arabidopsis, corn, wheat, soybean, and cotton.

For expression of a recombinant protein or peptide, or for modulation of an identified compensatory gene, any plant promoter can be used. A promoter may be a plant RNA polymerase II promoter. Elements included in plant promoters can be a TATA box or Goldberg-Hogness box, typically positioned approximately 25 to 35 basepairs upstream (5′) of the transcription initiation site, and the CCAAT box, located between 70 and 100 basepairs upstream. In plants, the CCAAT box may have a different consensus sequence than the functionally analogous sequence of mammalian promoters (Messing et al. (1983) In: Genetic Engineering of Plants, Kosuge et al., eds., pp. 211-227). In addition, virtually all promoters include additional upstream activating sequences or enhancers (Benoist and Chambon (1981) Nature 290:304-310; Gruss et al. (1981) Proc. Nat. Acad. Sci. 78:943-947; and Khoury and Gruss (1983) Cell 27:313-314) extending from around −100 bp to −1,000 bp or more upstream of the transcription initiation site.

Protein Production/fermentation

The process of the invention optimally leads to increased production of a disulfide bonded recombinant protein or peptide in a host cell expression system. The increased production alternatively can be an increased level of properly processed protein or peptide per gram of protein produced, or per gram of host protein. The increased production can also be an increased level of recoverable protein or peptide produced per gram of recombinant or per gram of host cell protein. The increased production can also be any combination of increased total level and increased properly processed, active or soluble level of protein.

The improved expression of recombinant protein can be an increase in the solubility of the protein. The protein or peptide can be insoluble or soluble. The protein or peptide can include one or more targeting sequences or sequences to assist purification.

Cell Growth

In some embodiments of the present invention, the cells can be grown in a media supplemented with redox cofactor. In these embodiments, the cofactor can be included in the media during cell growth, or can be added during the period of recombinant protein induction. The redox cofactor can also be added in increasing levels as a gradient during recombinant protein induction. In other embodiments, the cofactor can be included in the media only when recombinant protein production reaches a certain level, as measured by cell density or protein concentration.

The host cell can be transformed with at least one vector encoding the protein or peptide of interest. The host cell can also be transformed with nucleic acid sequences that encode a redox cofactor, or a precursor to a redox cofactor. Transformation of the host cells with the vector(s) may be performed using any transformation methodology known in the art, and the bacterial host cells may be transformed as intact cells or as protoplasts (i.e., including cytoplasts). Transformation methodologies can include poration methodologies, e.g., electroporation, protoplast fusion, bacterial conjugation, and divalent cation treatment, e.g., calcium chloride treatment or CaClMg2+ treatment, or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

As used herein, the term “fermentation” includes both embodiments in which literal fermentation is employed and embodiments in which other, non-fermentative culture modes are employed. Fermentation may be performed at any scale. In embodiments of the present invetion the fermentation medium may be selected from among rich media, minimal media, and mineral salts media. Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, B D Davis & E S Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. No organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. A mineral salts medium can contain glucose as the carbon source. In comparison to mineral salts media, minimal media can also contain mineral salts and a carbon source, but can be supplemented with, e.g., low levels of amino acids, vitamins, peptones, or other ingredients, though these are added at very minimal levels.

The expression system according to the present invention can be cultured in any fermentation format. For example, batch, fed-batch, semi-continuous, and continuous fermentation modes may be employed herein.

The expression systems according to the present invention are useful for transgene expression at any scale (i.e., volume) of fermentation. Thus, e.g., microliter-scale, centiliter scale, and deciliter scale fermentation volumes may be used; and 1 Liter scale and larger fermentation volumes can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In embodiments of the present invention, growth, culturing, and/or fermentation of the transformed host cells can be performed within a temperature range permitting survival of the host cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

Cell Density

When using Pseudomonas fluorescens in expressing recombinant disulfide bonded proteins, the cells can typically be grown in high cell densities compared to E. coli or other bacterial expression systems. To this end, Pseudomonas fluorescens expressions systems can provide a cell density of about 20 g/L or more. The Pseudomonas fluorescens expressions systems according to the present invention can likewise provide a cell density of at least about 70 g/L, as stated in terms of biomass per volume, the biomass being measured as dry cell weight.

In certain embodiments of the present invention the cell density can be at least 20 g/L. In other embodiments the cell density will be at least 25 g/L, 30 g/L, 35 g/L, 40 g/L, 45 g/L, 50 g/L, 60 g/L, 70 g/L, 80 g/L, 90 g/L, 100 g/L, 110 g/L, 120 g/L, 130 g/L, 140 g/L, or at least 150 g/L.

In other embodiments, the cell density at induction can be between 20 g/L and 150 g/L; 20 g/L and 120 g/L; 20 g/L and 80 g/L; 25 g/L and 80 g/L; 30 g/L and 80 g/L; 35 g/L and 80 g/L; 40 g/L and 80 g/L; 45 g/L and 80 g/L; 50 g/L and 80 g/L; 50 g/L and 75 g/L; 50 g/L and 70 g/L; 40 g/L and 80 g/L.

Isolation of Protein or Peptide of Interest

Generally, the process provides for an increase in the level of correctly or properly processed recombinant protein expressed, in comparison with conventional expression systems. In particular, the protein is not aggregated in the cytoplasm or periplasm, but is excreted into the extracellular media.

In some embodiments, the invention provides a process for improving the solubility of a recombinant protein or peptide in a host cell. The term “soluble” as used herein means that the protein is not precipitated by centrifugation at between approximately 5,000 and 20,000×gravity when spun for 10-30 minutes in a buffer under physiological conditions. Soluble proteins are not part of an inclusion body or other precipitated mass. Similarly, “insoluble” means that the protein or peptide that can be precipitated by centrifugation at between 5,000 and 20,000×gravity when spun for 10-30 minutes in a buffer under physiological conditions. Insoluble proteins or peptides can be part of an inclusion body or other precipitated mass. The term “inclusion body” is meant to include any intracellular body contained within a cell wherein an aggregate of proteins or peptides have been sequestered.

In certain embodiments of the present invention, no additional disulfide bond promoting conditions or agents are required in order to recover disulfide bond-containing identified polypeptide in active, soluble form from the host cell. Embodiments of the present invention can include the transgenic peptide, polypeptide, protein, or fragment thereof having a folded intramolecular conformation in its active state. In some embodiments, the transgenic peptide, polypeptide, protein, or fragment contains at least one intramolecular disulfide bond in its active state; and perhaps up to 2, 4, 6, 8, 10, 12, 14, 16, 18, or 20 or more disulfide bonds.

The proteins of this invention may be purified by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns. General techniques are further described in, for example, R. Scopes, Protein Purification: Principles and Practice, Springer-Verlag, N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); A K Patra et al., Protein Expr Purif, 18(2), p. 182-92 (2000); and R. Mukhija, et al., Gene 165(2), p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc., Chatsworth, Calif. Detection of the expressed protein can be achieved by methods known in the art and include, e.g., radioimmunoassays, Western blotting techniques or immunoprecipitation.

The recombinantly produced and expressed enzyme can be recovered and purified from the recombinant cell cultures by numerous methods, for example, high performance liquid chromatography (HPLC) can be employed for final purification steps, as necessary.

Certain proteins expressed in this invention may form insoluble aggregates (“inclusion bodies”). Several protocols are suitable for purification of proteins from inclusion bodies. For example, purification of inclusion bodies typically involves the extraction, separation and/or purification of inclusion bodies by disruption of the host cells, e.g., by incubation in a buffer of 50 mM TRIS/HCL pH 7.5, 50 mM NaCl, 5 mM MgCl₂,1 mM DTT, 0.1 mM ATP, and 1 mM PMSF. The cell suspension is typically lysed using two to three passages through a French Press. The cell suspension can also be homogenized using a Polytron (Brinkrnan Instruments) or sonicated on ice. Alternate methods of lysing bacteria are apparent to those of skill in the art (see, e.g., Sambrook et al., supra; Ausubel et al., supra).

If desired, the inclusion bodies can be solubilized, and the lysed cell suspension typically can be centrifuged to remove unwanted insoluble matter. Proteins that formed the inclusion bodies may be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art.

Alternatively, it is possible to purify the recombinant proteins or peptides from the host periplasm. After lysis of the host cell, when the recombinant protein is exported into the periplasm of the host cell, the periplasmic fraction of the bacteria can be isolated by cold osmotic shock in addition to other methods known to those skilled in the art. To isolate recombinant proteins from the periplasm, for example, the bacterial cells can be centrifuged to form a pellet. The pellet can be resuspended in a buffer containing 20% sucrose. To lyse the cells, the bacteria can be centrifuged and the pellet can be resuspended in ice-cold 5 mM MgSO₄ and kept in an ice bath for approximately 10 minutes. The cell suspension can be centrifuged and the supernatant decanted and saved. The recombinant proteins present in the supernatant can be separated from the host proteins by standard separation techniques well known to those of skill in the art.

An initial salt fractionation can separate many of the unwanted host cell proteins (or proteins derived from the cell culture media) from the recombinant protein of interest. One such example can be ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a recombinant protein can be used to isolated it from proteins of greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The recombinant protein will pass through the membrane into the filtrate. The filtrate can then be chromatographed as described below.

Recombinant proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Renaturation and Refolding

Insoluble protein can be renatured or refolded to generate secondary and tertiary protein structure conformation. Protein refolding steps can be used, as necessary, in completing configuration of the recombinant product. Refolding and renaturation can be accomplished using an agent that is known in the art to promote dissociation/association of proteins. For example, the protein can be incubated with dithiothreitol followed by incubation with oxidized glutathione disodium salt followed by incubation with a buffer containing a refolding agent such as urea.

Recombinant protein can also be renatured, for example, by dialyzing it against phosphate-buffered saline (PBS) or 50 mM Na-acetate, pH 6 buffer plus 200 mM NaCl. Alternatively, the protein can be refolded while immobilized on a column, such as the Ni NTA column by using a linear 6M-1M urea gradient in 500 mM NaCl, 20% glycerol, 20 mM Tris/HCl pH 7.4, containing protease inhibitors. The renaturation can be performed over a period of 1.5 hours or more. After renaturation the proteins can be eluted by the addition of 250 mM immidazole. Immidazole can be removed by a final dialyzing step against PBS or 50 mM sodium acetate pH 6 buffer plus 200 mM NaCl. The purified protein can be stored at 4° C. or frozen at −80° C.

Other methods include, for example, those that may be described in M. H. Lee et al., Protein Expr. Purif., 25(1), p. 166-73 (2002); W. K. Cho et al., J. Biotechnology, 77(2-3), p. 169-78 (2000); Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; S. Roe, Protein Purification Techniques: A Practical Approach (Practical Approach Series), Oxford Press (2001); D. Bollag, et al., Protein Methods, Wiley-Lisa, Inc. (1996).

Active Protein or Peptide Analysis

Active proteins can have a specific activity of at least 20%, 30%, or 40%, and preferably at least 50%, 60%, or 70%, and most preferably at least 80%, 90%, or 95% that of the native protein or peptide that the sequence is derived from. Further, the substrate specificity (k_{cat} /Kₘ) is optionally substantially similar to the native protein or peptide. Typically, k_{cat}/Kₘ will be at least 30%, 40%, or 50%, that of the native protein or peptide; and more preferably at least 60%, 70%, 80%, or 90%. Methods of assaying and quantifying measures of protein and peptide activity and substrate specificity (k_{cat}/Kₘ), are well known to those of skill in the art.

The invention can also improve recovery of active recombinant proteins or peptides. The activity of a recombinant protein or peptide produced in accordance with embodiments of the present invention can be measured by any protein specific conventional or standard in vitro or in vivo assay known in the art. The activity of the recombinant protein or peptide can be compared with the activity of the corresponding native protein to determine whether the recombinant protein exhibits substantially similar or equivalent activity to the activity generally observed in the native protein or peptide under the same or similar physiological conditions.

The activity of the recombinant protein can be compared with a previously established native protein or peptide standard activity. Alternatively, the activity of the recombinant protein or peptide can be determined in a simultaneous, or sunstantially simultaneous, comparative assay with the native protein or peptide. For example, in vitro assays can be used to determine any detectable interaction between a recombinant protein or peptide and a target, e.g., between an expressed enzyme and substrate, between expressed hormone and hormone receptor, between expressed antibody and antigen, etc. Such detection can include the measurement of calorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. In addition, in vivo assays include, but are not limited to, assays to detect physiological effects of the recombinant protein or peptide in comparison to physiological effects of the native protein or peptide, e.g., weight gain, change in electrolyte balance, change in blood clotting time, changes in clot dissolution and the induction of antigenic response. Generally, any in vitro or in vivo assay can be used to determine the active nature of the recombinant protein or peptide that allows for a comparative analysis to the native protein or peptide so long as such activity is assayable. Alternatively, the proteins or peptides produced in the present invention can be assayed for the ability to stimulate or inhibit interaction between the protein or peptide and a molecule that normally interacts with the protein or peptide, e.g., a substrate or a component of the signal pathway that the native protein normally interacts. Such assays can typically include the steps of combining the protein with a substrate molecule under conditions that allow the protein or peptide to interact with the target molecule, and detect the biochemical consequence of the interaction with the protein and the target molecule.

Assays that can be utilized to determine protein or peptide activity are described, for example, in Ralph, P. J., et al. (1984) J. Immunol. 132:1858, or Saiki et al. (1981) J. Immunol. 127:1044; Steward, W. E. 11 (1980) The Interferon Systems, Springer-Verlag, Vienna and New York; Broxmeyer, H. E., et al. (1982) Blood 60:595; Molecular Cloning: A Laboratory Manual, 2d ed., Cold Spring Harbor Laboratory Press, Sambrook, J., E. F. Fritsch and T. Maniatis eds., 1989; and Methods in Enzymology: Guide to Molecular Cloning Techniques, Academic Press, Berger, S. L. and A. R. Kimmel eds., 1987; A. K. Patra et al., Protein Expr Purif, 18(2), p. 182-92 (2000); Kodama et al., J. Biochem. 99:1465-1472 (1986); Stewart et al., Proc. Nat'l Acad. Sci. USA 90:5209-5213 (1993); Lombillo et al., J. Cell Biol. 128:107-115 (1995); Vale et al., Cell 42:39-50 (1985). Generally, any in vivo assay can be used so long as a variable parameter exists so as to detect a change in the interaction between the identified and the polypeptide of interest. See, for example, U.S. Pat. No. 5,834,250.

If overexpression of a gene coding a protein disulfide bond isomerase or pathway of isomerases and accessory proteins does not lead to more efficient.folding and disulfide bond formation in the heterologous protein it may be because the availability of the redox cofactor has become rate limiting to the reaction. Therefore, overexpression of the isomerases only leads to the production of inactive protein. It may be necessary not only to over-express the isomerases, but also derepress or unregulate to some extent the pathway(s) that supply the redox cofactors to the isomerases. Doing both may lead to a real enhancement of the folding and disulfide bond formation pathways in microbes like Escherichia coli or Pseudomonas fluorescens. This enhancement would lead to the ability to recover more active heterologous, disulfide-bonded protein from these organisms and reduce the cost of their production.

In order that this invention may be better understood, the following examples are set forth. These examples are for purposes of illustration only, and are not to be construed as limiting the scope of the invention in any manner.

### EXAMPLES

### Example 1

### Disulfide Bond Formation in Gram Negative Bacteria

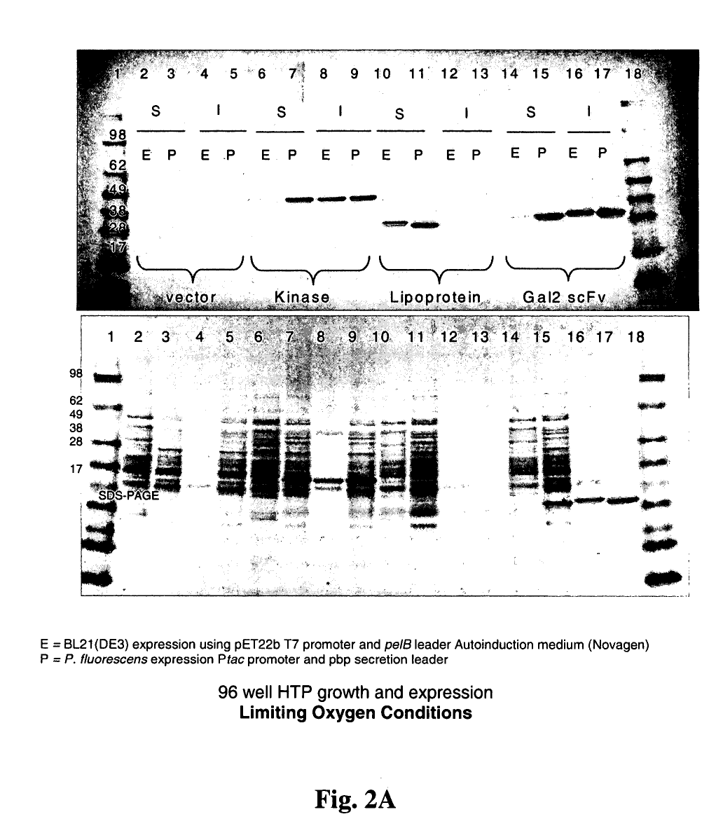

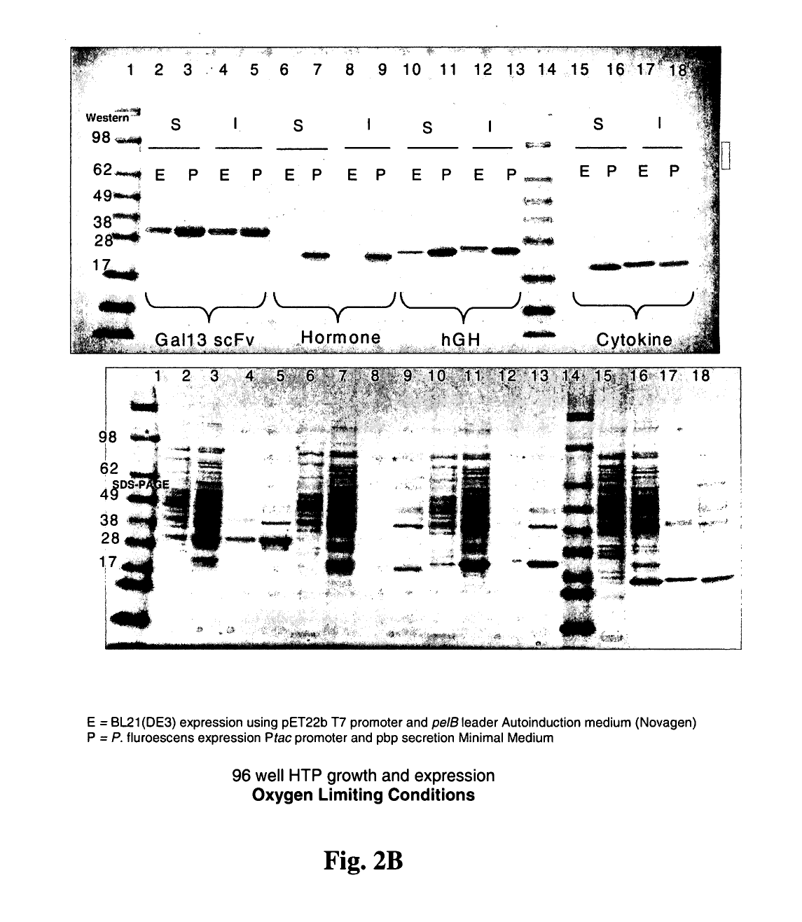

Heterologous gene induction in P. fluorescens fermentations is done under conditions when oxygen is not limited. As an obligate aerobe, this organism will not grow or produce ATP or GTP (necessary for cellular metabolism including protein biosynthesis) unless oxygen is present in the growth medium. As shown in FIGS. 2A and 2B, cells were cultured in 96-well plates with shaking, micro-aerophilic conditions, where E=E. coli, P=P. fluorescens, S=soluble cell fraction, and I=insoluble cell fraction. Because electrons resulting from the oxidation of protein disulfide bonds (DsbA, DsbB catalyzed rections) are passed directly into the pathway of oxidative phosphorylation, this pathway for the oxidation of sulfhydriles will always be available to P. fluorescens in an oxidative growth environment, even if those conditions are microaerophilic. E. coli on the other hand, as a facultative aerobe, tends to begin growing anaerobically and shuts down the oxydative phosphorylation pathway in microaerophilic conditions. Because of this, it is predicted that E. coli should be disadvantaged for the expression and folding (disulfide bond formation) when grown under microaerophilic conditions as compared to P. fluorescens. Seven different human or other heterologous proteins containing multiple disulfide bonds were expressed in either E. coli or P. fluorescens. These proteins were expressed to be secreted into the periplasmic space of both organisms. In the case of E. coli, the secretion leader peptide was pelB in all cases. For the P. fluorescens constructs, it was the native pbp (phosphate binding protein) leader sequence in all cases. Using best practices for expression in both cases with expression conditions done in a 96 well shaken plate format, it is seen that secreted expression of all proteins in P. fluorescens is advantaged over E. coli either in the amount or solubility (native conformation) of the recombinant proteins. This example shows that aeorophic, even microaerophilic conditions of growth, favor formation of disulfide bonds. These results show that having an intact redox pathway through to a terminal electron acceptor, oxygen, and all of its components favors disulfide bond formation in over-expressed heterologous proteins.

### Example 2

### Disulfide Bond Formation in Gram Negative Bacteria

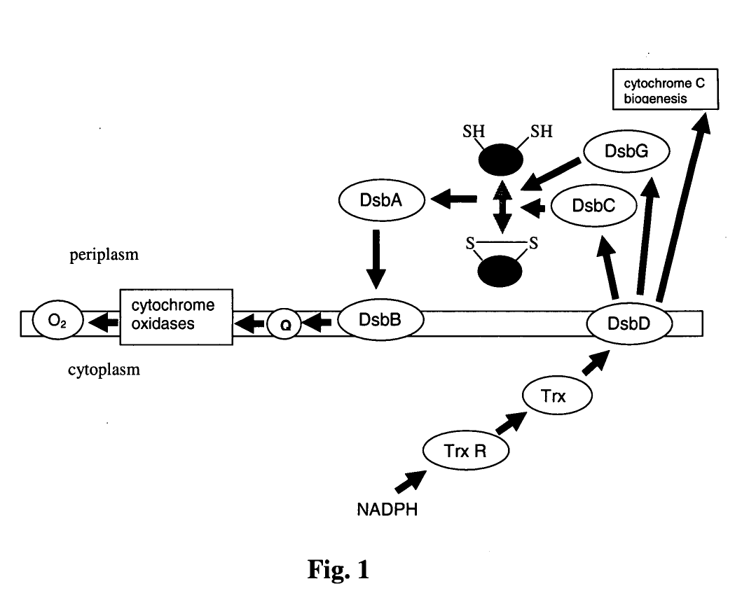

The quinone reductase activity of DsbB involves two quinones: a prosthetic quinone, which remains bound to DsbB, and a transient (diffusible substrate) quinone. (Regeimbal, J., S. Gleiter, B. L. Trumpower, C.-A. Yu, M. Diwakar, D. P. Ballou, and J. C. A. Bardwell. 2003. Disulfide bond formation involves a quinhydrone-type charge-transfer complex. PNAS 100:13779-13784). The prosthetic and transient quinones are initially reduced. An oxidized quinone derived from the quinone pool (electron transport chain) replaces the transient reduced quinone. The resident reduced quinone then transfers electrons to the transient quinone through a quinhydrone complex. The reoxidation of DsbA results in reduction of the C-terminal disulfide in DsbB which undergoes dithiol-disulfide exchange with the N-terminal cysteines. The newly formed N-terminal dithiol then reduces the prosthetic quinine. Because the two reduced quinones do not form a stable complex, the transient hydroquinone is exchanged with an oxidized quinone. At this state, DsbB can enter a new cycle of catalysis to reoxidize another molecule of DsbA. This means that quantitative amounts of oxidized quinone are required to keep DsbA in the oxidized state. Therefore, one oxidized quinone is required to create each disulfide bond in a target protein. Thus, without a one to one stoichiometry of quinone to DsbB, the enzyme will be completely inactive. Therefore, overproduction of DsbB or any of the other enzymes by recombinant means of the pathway depicted in FIG. 1 cannot be expected, on on its own, to result in an improvement in the cell's ability to catalyze the formation of disulfide bonds. Evidence that modulation of the pool of quinone is also required is interpreted from Example 3.

### Example 3

### Reduced Availability of Quinone Cofactor Reduces the Ability of Gram Negative Bacterium to Produce Disulfide Bonds in Heterologously Expressed Proteins

The water-soluble thiols 2-mercaptoethanol, 1-thioglycerol, and dithiothreitol inhibit gram-positive and gram-negative bacteria at millimolar concentrations (Zeng, H., I. Snavely, P. Zamorano, and G. T. Javor, 1998, Low Ubiquinone Content in Escherichia coli Causes Thiol Hypersensitivity, J. Bacteriol. 180:3681-3685). Several processes are affected, and include interference with the formation of disulfide bonds of periplasmic and outer membrane proteins. In an attempt to look for genes which may be regulating these responses, Zeng et al. searched for thiol-hypersensitive mutants of E. coli. (Id.) The search was based on the rationale that should such a gene(s) exist, their inactivation would likely yield a thiol-hypersensitive phenotype. Zeng et al mutagenized the THU (ubiX) strain of E. coli. The cells were grown on minimal glucose plates containing 25 mM 1-thioglycerol. The smallest colonies were replica plated onto minimal medium- and minimal medium-plus-25 mM-1-thioglycerol. The isolate exhibiting the greatest hypersensitivity to 1-thioglycerol, named IS16, was used for further studies. Strain IS16 was determined to be a ubiX ubiD double mutant. These alleles code for isozymes of the p-hydroxybenzoate decarboxylase a step in the pathway of quinone biosynthesis. Therefore, this pathway is completely blocked at that step in this double mutant strain, and the strain will not synthesize quinone.

Strains IS16 (ubiX ubiD), IS16B1 (IS16 with ubiX on a plasmid), and AN385 (ubiA) were tested for any evidence of the Dsb phenotype. The test was the induction of the periplasmic enzyme, alkaline phosphatase, which needs two disulfide bonds for activity. Strains IS16 (ubiX) and AN385 (ubiA) could not produce functional alkaline phosphtase within 120 minutes of induction. In contrast, the parent strains and strain IS16B1 began making alkaline phosphatase within an hour. It was concluded that the low ubiquinone-containing strains exhibited a Dsb-negative phenotype. This experiment demonstrates that the cell's ability to form disulfide bonds in the periplasmic space can be strongly limited by the availability of quinone cofactor.

A recent report suggests that there is a direct link between the respiratory chain and the DsbA-DsbB disulfide bond-forming system (Kobayashi, T., S. Kishigami, M. Sone, H. Inokuchi, T. Mogi, and K. Ito. 1997). Respiratory chain is required to maintain oxidized states of the DsbA-DsbB disulfide bond formation system in aerobically growing Escherichia coli cells. (PNAS 94:11857-11862.) Electrons removed from the periplasmic cysteine residues during disulfide bond formation pass first to the DsbA protein, then to the cytoplasmic membrane-associated DsbB protein, and finally to the respiratory chain. In support of their thesis, the authors showed that a ubiA menA double mutant, when deprived of para-hydroxybenzoate, slowed its growth (presumably because of reduction of ubiquinone content) and accumulated first reduced forms of DsbA and DsbB proteins and then the DsbA-DsbB complex. This finding could explain how continually low ubiquinone levels, such as those seen in strains IS16 and AN385, would reduce the ability of the respiratory chain to accept electrons from the DsbA-DsbB complex. Accumulation of reduced DsbA and DsbB proteins and the DsbA-DsbB complex, in turn, would result in a Dsb phenotype.

### Example 4

### Over-Expression of DsbA and DsbC Lacking Improved Expression of Heterologous, Disulfide Bonded Protein in E. coli

The ability of E. coli to over express and correctly fold recombinant proteins containing multiple disulfide bonds is limited. Most E. coli periplasmic and outer membrane proteins contain two or fewer disulfides per monomer. To boost the endogenous capacity of the wild-type cells to accomplish this task, overexpression of the DsbA and DsbC proteins is often done to get disulfides to form more efficiently in recombinant proteins. Joly et al. (5) have found that transient overexpression of either DsbA or DsbC resulted in a large yield increase of recombinant insulin-like growth factor I (IGF-I) in E. coli. (Joly, J. C., W. S. Leung, and J. R. Swartz, 1998, Overexpression of Escherichia coli oxidoreductases increases recombinant insulin-like growth factor-I accumulation, PNAS 95:2773-2777.) However, this increase was not accompanied by an increase in the amount of folded, disulfide-bonded protein. These authors hypothesized that elevated concentrations of disulfide bond-forming enzymes would improve IGF-I folding. Surprisingly, it was discovered that overexpression of these enzymes led to increased aggregation and higher yields of IGF-I. Given these results, they further hypothesized that there must be a limitation in the system for maintaining DsbA in the oxidized state. They also state that, “the source of the limitation is currently unknown.” (Id.) Of course DsbB is responsible for keeping DsbA in the oxidized state, which is required to oxidize reduced thiols in unfolded proteins. It is possible that the failure of DsbA to remain oxidized is a direct result of a limitation of oxidized quinone given that the system is overtaxed by the very high level of expression (reported the level was 30% of the total cell protein) of the recombinant IGF-1.

We interpret the evidence presented in these four examples in a novel way. Namely, that the failure of bacterial strains that have been engineered to over-express the enzymes of the disulfide bond formation pathway, in order to facilitate disulfide bond formation in highly overexpressed heterologous proteins, often fail to improve the extent of properly disulfide bonded protein produced, not for lack of the enzyme itself, but for lack of the cofactor, quinone, required in quantitative amounts to accomplish the catalysis. This means that overproduction of the redox cofactor must accompany over-expression of the enzymes for there to be complete and efficient disulfide bond formation in highly over-expressed recombinant proteins, like the IGF-1 case cited in the present example.

What is claimed is:

1. A process for producing in a bacterial cell, a biologically-active, soluble polypeptide having at least one disulfide bond, comprising expressing in said cell a first DNA segment encoding a disulfide bond isomerase or disulfide bond oxido-reductase/isomerase pair operably linked to a secretion signal sequence and a second DNA segment encoding said polypeptide operably linked to a secretion signal sequence under conditions effective to produce said polypeptides.

2. The process according to claim 1, wherein said polypeptide is a eukaryotic or prokaryotic peptide.

3. The process according to claim 1, wherein the bacterial cell is derived from Pseudomonas fluorescens.

4. The process according to claim 1, further comprising increasing a redox cofactor.

5. The process according to claim 4, further comprising increasing expression of at least one gene that is involved in a redox cofactor or a metabolic precursor of a redox cofactor biosynthesis.

6. The process according to claim 1, further comprising culturing the bacterial cell in a fermentor under conditions effective to produce said polypeptides.

7. The process according to claim 1, further comprising elevating the intracellular concentration of a redox cofactor or a metabolic precursor of a redox cofactor through its addition to the culture growth medium.

8. An expression system for producing in a bacterial cell, a biologically-active, soluble eukaryotic polypeptide, said system comprising a first DNA segment and a second DNA segment, wherein said first segment encodes a disulfide isomerase or isomerase/oxido-reductase pair and said second segment encodes a polypeptide having at least one disulfide bond.

9. The expression system of claim 8, wherein said polypeptide is a eukaryotic or prokaryotic polypeptide.

10. The process according to claim 8, further comprising elevating the biosynthetic rate and consequently the intracellular concentration of a redox cofactor.

11. The process according to claim 10, further comprising increasing expression of gene or genes that are involved in a redox cofactor or a metabolic precursor of a redox cofactor biosynthesis.

12. The expression system of claim 8, wherein the redox cofactor is induced in the cell from a recombinant DNA.

13. The expression system of claim 8, wherein the cell is derived from Pseudomonas fluorescens.

14. A process for increasing the formation of the natural protein conformation when disulfide bonded proteins are secreted by an Pseudomonas fluorescens host cell that contains a recombinant DNA coding for the secreted protein, the process comprising culturing the host in a suitable culture medium for the expression of the recombinant DNA, wherein said culture medium contains 0.1 to 20 mmol/l of one or more thiol reagents.

15. The process according to claim 14, wherein the formation of the natural protein conformation is further increased by increasing a redox cofactor.

16. The process according to claim 15, further comprising increasing expression of a gene that is involved in cofactor biosynthesis.

17. The process according to claim 15, further comprising increasing expression of gene or genes that are involved in a redox cofactor or a metabolic precursor of a redox cofactor biosynthesis.

18. A recombinant vector comprising a first transcriptional unit encoding a protein disulfide isomerase or disulfide isomerase/oxidoreductase pair operably linked to a first signal sequence and said second transcriptional unit comprises a DNA segment encoding a polypeptide having at least one disulfide bond operably linked to a second signal sequence.

19. The recombinant vector according to claim 18, wherein said polypeptide is a eukaryotic or prokaryotic polypeptide.

20. The recombinant vector according to claim 19, wherein said eukaryotic polypeptide is a mammalian polypeptide.

21. The recombinant vector according to claim 18, wherein said protein disulfide isomerase or disulfide isomerase/oxidoreductase pair is a bacteria, rat, yeast, or human protein disulfide isomerase or isomerase/oxidoreductase pair.

22. A prokaryotic cell transformed with the recombinant vector according to claim 18.

23. The cell of claim 22, wherein the cell is Pseudomonas fluorescens.
